SEQUENCE LISTING (28 sequences)

SEQ ID NO: 1 - DNA, 321 bp, Artificial Sequence
  Synthetic h2L
  CDS (1)..(321)
gatatccaactgacccagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccacgttcggtgctgggaccaagctggagatcaaa

SEQ ID NO: 2 - protein, 107 aa, Artificial Sequence
  Synthetic Construct
DIQLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLEIK

SEQ ID NO: 3 - DNA, 357 bp, Artificial Sequence
  Synthetic h2H
  CDS (1)..(357)
caggtacaactgcaggagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggaccacggtcaccgtttcctcg

SEQ ID NO: 4 - protein, 119 aa, Artificial Sequence
  Synthetic Construct
QVQLQESGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTTVTVSS

SEQ ID NO: 5 - DNA, 321 bp, Artificial Sequence
  Synthetic chimeric Sequence (hOL)
  CDS (1)..(321)
gatatccagatgacccagagcccgagctctctgagcgcgagcgtgggcgatcgcgtgaccattacgtgcagcgcgtctagctctgtgagctatatgaactggtaccagcaaaccccaggcaaagcgccgaaacgctggatttatgataccagcaaactggcgagcggcgtgccgagccgctttagcggctctggtagcggcaccgattatacgtttaccattagctctctgcagccggaagatattgcgacctattactgccagcaatggagctctaacccgtttacctttggccagggtaccaaactgcagattacccgc

SEQ ID NO: 6 - protein, 107 aa, Artificial Sequence
  Synthetic Construct (hOL)
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 7 - DNA, 357 bp, Artificial Sequence
  Synthetic chimeric Sequence (hOH)
  CDS (1)..(357)
caggtgcaactggtgcagagcggcggtggcgttgtgcagccgggccgcagcctgcgcctgtcttgcaaagcgagcggctatacctttacgcgctataccatgcattgggtgcgccaggcgccgggcaaaggtctggaatggattggctatattaacccgtctcgcggctataccaactataatcagaaagtgaaagatcgctttaccattagccgcgataactctaaaaacaccgcgtttctgcagatggatagcctgcgcccggaagataccggcgtgtatttttgcgcgcgctactatgatgaccattatagcctggattattggggccagggcaccccggtgaccgttagctcg

SEQ ID NO: 8 - protein, 119 aa, Artificial Sequence
  Synthetic Construct (hOH)
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 9 - DNA, 51 bp, Artificial Sequence
  Synthetic back primer (NcoI-225H)
  misc_feature (1)..(3): n is a, c, g, or t
nnnccatggcccaggtacaactgcaggagtcaggacctggcctagtgcagc

SEQ ID NO: 10 - DNA, 46 bp, Artificial Sequence
  Synthetic forward primer (225H-EagI)
  misc_feature (1)..(3): n is a, c, g, or t
nnncggccgaggaaacggtgaccgtggtcccttggccccagtaagc

SEQ ID NO: 11 - DNA, 24 bp, Artificial Sequence
  Synthetic back primer (EcoRV-225L)
  misc_feature (1)..(3): n is a, c, g, or t
nnngatatccaactgacccagtct

SEQ ID NO: 12 - DNA, 34 bp, Artificial Sequence
  Synthetic forward primer (225L-SacII)
  misc_feature (1)..(3): n is a, c, g, or t
nnnccgcggcacgtttgatctccagcttggtccc

SEQ ID NO: 13 - DNA, 39 bp, Artificial Sequence
  Synthetic back primer (SpeI-pelB)
  misc_feature (1)..(3): n is a, c, g, or t
nnnactagttatttcaaggagacagtcataatgaaatac

SEQ ID NO: 14 - DNA, 34 bp, Artificial Sequence
  Synthetic forward primer (T7term-EcoRI)
  misc_feature (1)..(3): n is a, c, g, or t
nnngaattcatccggatatagttcctcctttcag

SEQ ID NO: 15 - DNA, 77 bp, Artificial Sequence
  Synthetic forward primer (225H-G3-EcoRV)
  misc_feature (1)..(3): n is a, c, g, or t
nnngatatcggatccgccaccgcccgacccgccaccgccgctaccgcccccgccggccgaggaaacggtgaccgtgg

SEQ ID NO: 16 - DNA, 43 bp, Artificial Sequence
  Synthetic back primer (NcoI-2L)
  misc_feature (1)..(3): n is a, c, g, or t
nnnccatggccgatatccaactgacccagtctccagtcatcct

SEQ ID NO: 17 - DNA, 50 bp, Artificial Sequence
  Synthetic forward primer (2L-G1-OH)
acctggccaccgccaccagatttgatctccagcttggtcccagcaccgaa

SEQ ID NO: 18 - DNA, 40 bp, Artificial Sequence
  Synthetic back primer (2L-G1-OH)
agatcaaatctggtggcggtggccaggtgcaactggtgca

SEQ ID NO: 19 - DNA, 40 bp, Artificial Sequence
  foraward primer (OH-SacII)
  misc_feature (1)..(4): n is a, c, g, or t
nnnnagccgcggagctaacggtcaccggggtgccctggcc

SEQ ID NO: 20 - DNA, 36 bp, Artificial Sequence
  Synthetic back primer (NcoI-OL)
  misc_feature (1)..(4): n is a, c, g, or t
nnnnccatggccgatattcagatgacccagagcccg

SEQ ID NO: 21 - DNA, 41 bp, Artificial Sequence
  Synthetic forward primer (OL-G1-2H)
ttgtacctggccaccgccaccagaggtaatctgcagtttgg

SEQ ID NO: 22 - DNA, 48 bp, Artificial Sequence
  Synthetic back primer (OL-G1-2H)
attacctctggtggcggtggccaggtacaactgcaggagtcaggacct

SEQ ID NO: 23 - DNA, 31 bp, Artificial Sequence
  Synthetic forward primer (2H-SacII)
  misc_feature (1)..(4): n is a, c, g, or t
nnnnccgcggaggaaacggtgaccgtggtcc

SEQ ID NO: 24 - DNA, 36 bp, Artificial Sequence
  Synthetic back primer (NcoI-5L)
  misc_feature (1)..(4): n is a, c, g, or t
nnnnccatggccgatattgtgatgacccagagcccg

SEQ ID NO: 25 - DNA, 37 bp, Artificial Sequence
  Synthetic forward primer (OL-Sac-II)
  misc_feature (1)..(4): n is a, c, g, or t
nnnnagccgcggcgcgggtaatctgcagtttggtacc

SEQ ID NO: 26 - DNA, 68 bp, Artificial Sequence
  Synthetic back primer (2L-G3-2H)
aaaggcgggggcggtagcggcggtggcgggtcgggcggtggcggatcccaggtacaactgcaggagtc

SEQ ID NO: 27 - DNA, 68 bp, Artificial Sequence
  Synthetic forward primer (2L-G3-2H)
ctgggatccgccaccgcccgacccgccaccgccgctaccgcccccgcctttgatctccagcttggtcc

SEQ ID NO: 28 - DNA, 33 bp, Artificial Sequence
  Synthetic back primer (2H-G1-OH)
gtttcctccggcgggggcggttcgcaggtgcaa

(19) United States
(12) Patent Application Publication
(10) Pub. No.: US 2017/0274072 A1
(43) Pub. Date: Sep. 28, 2017

(54) BISPECIFIC ANTIBODY TARGETING HUMAN EPIDERMAL GROWTH FACTOR RECEPTOR

(71) Applicant: TOHOKU UNIVERSITY, Sendai-shi, Miyagi (JP)

(72) Inventors: Izumi KUMAGAI, Sendai-shi (JP); Ryutaro ASANO, Sendai-shi (JP); Mitsuo UMETSU, Sendai-shi (JP)

(73) Assignee: TOHOKU UNIVERSITY, Sendai-shi, Miyagi (JP)

(21) Appl. No.: 15/310,358

(22) PCT Filed: Feb. 25, 2015
(86) PCT No.: PCT/JP2015/055357

§ 371 Date: Jun. 12, 2017

(30) Foreign Application Priority Data
Mar. 26, 2014 (JP) 2014-064141

(51) Int. Cl. A61K 39/395; C07K 16/40; C07K 16/28

(52) U.S. Cl. CPC A61K 39/39558; C07K 16/2863; C07K 16/40; C07K 16/2809; A61K 2039/507

(57) ABSTRACT

To provide bispecific antibodies and highly functional bispecific antibodies, which are produced by using a new anti-human EGF receptor 1 (Her 1) antibody different from the antibody 528. A bispecific antibody, comprising a variable region of the light chain (2L: SEQ ID NO:2) and a variable region of the heavy chain (2H: SEQ ID NO:4) of an anti-human EGF receptor 1 antibody 225, and a humanized variable region of the light chain (OL: SEQ ID NO:6) and a humanized variable region of the heavy chain (OH: SEQ ID NO:8) of an anti-CD3 antibody OKT, and the like.

### FIELD OF THE INVENTION

The present invention is related to a bispecific antibody, which is superior in stability and can be used in a cancer-specific immunotherapy, a single-chain polypeptide constituting the antibody, a nucleic acid encoding the polypeptide, a method for the production of the antibody, use of them as a pharmaceutical preparation, etc.

### BACKGROUND OF THE INVENTION

Recently, immunotherapy has been used as a safe therapy for the treatment of cancer, rheumatoid, etc. An antibody showing a cancer-specific cytotoxic activity (cytotoxicity) is used in the immunotherapy of cancer. While it is recognized that an antibody drug comprising such antibody will show high and safe therapeutic effects with little side effects, it has a problem that it would cost much since said drug needs to be produced by using established animal cells.

As a result, the production of recombinant antibodies having an extremely strong activity has been tried as a means for extremely reducing a dosage so as to cut cost.

Among these recombinant antibodies, an antibody with bispecificity (Bispecific Antibody: BsAb) has been studied intensively. This is because the bispecific antibody can bind specifically to two different kinds of antigens so that it will be utilized as a therapeutic agent having a specific anti-cancer effect. A diabody (Db) is a minimum unit of the above bispecific antibody. It was developed by utilizing the property that the variable region in a heavy chain (VH) and the variable region in a light chain (VL) originated from the same parent antibody will form a hetero-dimer through a non-covalent bond (Non-Patent Document 1). Methods for the production of bispecific antibodies other than the diabody-type bispecific antibody are described in Non-Patent Documents 2 and 3.

The present inventors already found that the diabody-type bispecific antibody (Ex3) that was produced by utilizing an anti-human epidermal growth factor (EGF) receptor 1 (Her 1) antibody 528 and an anti-CD3 antibody OKT3, and its humanized diabody-type bispecific antibody (referred to as “hEx3” in Patent Document 1) showed extremely strong anti-tumor effects. Furthermore, the present inventors have developed a highly functional bispecific antibody having various structures, based on said humanized diabody-type bispecific antibody (Patent Document 2).

The present inventors have also developed a LH-type bispecific antibody that is characterized in that the variable region of a light chain is located at an N-end (N-terminal) of each polypeptide constituting the humanized diabody-type bispecific antibody, and a humanized highly functional bispecific antibody comprising said LH-type bispecific antibody (Patent Document 3). They have further developed antibodies wherein the heavy or light chain of the Her 1 antibody 528 has various kinds of mutation/substitution of amino acid(s) (Patent Documents 4 and 5).

The highly functional bispecific antibodies disclosed in Patent Documents 2-5 are bispecific antibodies that comprise an Fc region in addition to the variable regions of the light and heavy chains of the anti-human EGF receptor 1 antibody 528 and the anti-CD3 antibody OKT3. These humanized highly functional bispecific antibodies have a significantly increased cytotoxicity when compared with Ex3, and a divalent binding activity for each antigen. A bispecific antibody with a minimized additional sequence such as Tag may be easily prepared by digestion of the above humanized highly functional bispecific antibodies with a protease, and easily purified with Protein A. They may be further provided with an effector property such as induction of an antibody-dependent cellular cytotoxicity (ADCC) activity and a complement-dependent cytotoxicity (CDC) function.

### RELATED ARTS

### Patent Document

Patent Document 1: Japanese Patent No. 3803790

Patent Document 2: WO 2007/108152 A1

Patent Document 3: WO 2010/109924 A1

Patent Document 4: WO 2011/062112 A1

Patent Document 5: WO 2012/020622 A1

### Non-Patent Document

Non-Patent Document 1: Hollinger, et al., Proc. Natl. Acad. Sci. USA 90, 6444-6448, 1993

Non-Patent Document 2: Alt M, et. al. Novel tetravalent and bispecific IgG-like antibody molecules combining single-chain diabodies with the immunoglobulin gammal Fc or CH3 region. FEBS Lett., 454, 90-4. (1999)

Non-Patent Document 3: Lu D, et. al. A fully human recombinant IgG-like bispecific antibody to both the epidermal growth factor receptor and the insulin-like growth factor receptor for enhanced antitumor activity. J Biol Chem., 280, 19665-72. (2005)

Non-Patent Document 4: J Biol Chem, 2005:280 (20) 19665-72

### DISCLOSURE OF THE INVENTION

### Problem to be Solved by the Invention

Although various kinds of the above bispecific antibodies (BsAb) have extremely excellent properties, there is a risk that some of them may drop out in the course of development of a final drug comprising them due to various reasons. In order to prepare for such cases, it is required to develop a diabody-type bispecific antibody and various kinds of highly functional bispecific antibody comprising a constituent derived from variable regions of a different anti-human EGF receptor 1 (Her 1) antibody from the antibody 528.

The problem to be solved by the present invention is therefore to provide bispecific antibodies and highly functional bispecific antibodies, which are produced by using a new anti-human EGF receptor 1 (Her 1) antibody different from the antibody 528.

### Means for Solving the Problems

The present inventors have succeeded in the production of a bispecific antibody by using an antibody 225 instead of the antibody 528, which shows excellent properties such as higher anti-tumor effects, leading to the accomplishment of the present invention.

The present invention is therefore related to the following aspects:

[1] A bispecific antibody, comprising a variable region of the light chain (2L: SEQ ID NO:2) and a variable region of the heavy chain (2H: SEQ ID NO:4) of an anti-human EGF receptor 1 antibody 225, and a humanized variable region of the light chain (OL: SEQ ID NO:6) and a humanized variable region of the heavy chain (OH: SEQ ID NO:8) of an anti-CD3 antibody OKT.

[2] The antibody according to the aspect [1], which is a diabody-type bispecific antibody.

[3] The antibody according to the aspect [1] or [2], wherein the variable region of the light chain is located at an N-end side of the variable region of the heavy chain (LH-type) in each polypeptide.

[4] The antibody according to the aspect [1], which is a tandem-type single chain antibody (scFv) having the structure represented by (2L2H)-(peptide linker)-(OHOL)

[5] The antibody according to the aspect [1], which further comprises a hinge region and Fc region.

[6] The antibody according to any one of the aspects [1]-[5], which is a bispecific antibody for human EGF receptor 1 and CD3.

[7] A single-chain polypeptide constituting the antibody of any one of the aspects [1]-[6].

[8] A nucleic acid molecule encoding the polypeptide of the aspect [7].

[9] A replicable cloning vector or an expression vector containing the nucleic acid molecule of the aspect [8].

[10] The vector of the aspect [9], which is a plasmid vector.

[11] A host cell transformed with the vector of the aspect [9] or [10].

[12] The hose cell of the aspect [11], which is a mammalian cell.

[13] A method for the production of the antibody of any one of the aspects [1]-[6], comprising culturing a host cell according to the aspect [11] to express the nucleic acid molecule, and collecting and purifying the single-chain polypeptides according to the aspect [7], assembling the resulting single-chain polypeptides, and separating and collecting the antibody thus formed.

[14] A pharmaceutical composition comprising the antibody of any one of the aspects [1]-[6] as an active ingredient.

[15] The pharmaceutical composition of the aspect [14] for use in eliminating, hurting, damaging and/or reducing tumor cells.

### ADVANTAGES OF THE INVENTION

As seen from the Examples mentioned below, the bispecific antibody according to the present invention, especially, an LH-type of the diabody-type bispecific antibody, showed excellent effects in cytotoxicity activity, capability of inducing cytokine secretion, anti-tumor activity in vivo using cancer-harboring mouse, and the like.

It is known that the antibodies 528 and 225 will show binding-inhibition against each other (Mol Biol Med. 1983; 1(5)511-29), and treatment test using a mouse showed that they had equivalent effects (Cancer Res. 1993; 53 (19)4637-42). However, it was confirmed that the LH-type bispecific antibody according to the present invention had an extremely higher activity than the LH-type bispecific antibody (Patent Document 3) having as a constituent the variable region derived from 528 antibody.

### BRIEF DESCRIPTION OF THE DRAWINGS

FIG. 1 shows HL-type co-expression vector, (b) LH-type co-expression vector, and (c) tandem-type scFv expression vector.

FIG. 2 shows the results of evaluation of cytotoxicity of E₂₂₅x3 prepared from an insoluble fraction.

FIG. 3 shows the results of evaluation of cytotoxicity (comparison between E₂₂₅x3 and Ex3).

FIG. 4 shows the orientation of E₂₂₅x3 Db.

FIG. 5 shows the construction of pRA1-E₂₂₅x3 O2G1.

FIG. 6 shows the construction of pRA1-E₂₂₅x3 2OG1.

FIG. 7 shows the results of purification of various E₂₂₅x3 Dbs with gel-filtration chromatography.

FIG. 8 shows the comparison of various E₂₂₅x3 Dbs in cytotoxicity.

FIG. 9 shows the results of SPR determination of various E₂₂₅x3 Dbs for EGFR.

FIG. 10 shows the comparison of various E₂₂₅x3 Dbs in bridging capability: a (A431, E₂₂₅x3 Db, CD3-FITC), b(T-LAK, E₂₂₅x3 Db, EGFR-FITC).

FIG. 11 shows the comparison of various E₂₂₅x3 Dbs in secretion of IFN-γ (upper) and TNF-α (lower).

FIG. 12 shows the comparison of cytotoxicity of Ex3 Db and E₂₂₅x3 Db by MTS assay.

FIG. 13 shows the comparison of cytotoxicity of Ex3 LHG1 and E₂₂₅x3 LHG1.

FIG. 14 shows scheme of tumor early stage model and tumor establish model.

FIG. 15 shows the results of evaluation of in vivo activity (tumor early stage model) of Ex3 LHG1 and E₂₂₅x3 LHG1.

### BEST MODE FOR CARRYING OUT THE INVENTION

A first aspect of the present invention relates to a bispecific antibody, comprising a variable region of the light chain (2L: SEQ ID NO:2) and a variable region of the heavy chain (2H: SEQ ID NO:4) of an anti-human EGF receptor 1 antibody 225, and a humanized variable region of the light chain (OL: SEQ ID NO:6) and a humanized variable region of the heavy chain (OH: SEQ ID NO:8) of an anti-CD3 antibody OKT. As a result, the antibody according to the present invention has a bi-specificity for human EGF receptor 1 and CD3.

A diabody-type bispecific antibody named as “E₂₂₅x3” may be mentioned as a representative example of the above bispecific antibody. An LH-type of the diabody-type bispecific antibody wherein the variable region of the light chain is located at an N-end side of the variable region of the heavy chain (LH-type) in each polypeptide constituting the bispecific antibody is preferable since it can show more excellent effects.

The sequence of the variable regions of the anti-human EGF receptor 1 antibody 225 is known, as shown such as in Int J Cancer. 1995 Jan. 3; 60 (1)137-44.

Specifically, base (nucleotide) sequences of the variable region of the light chain (SEQ ID NO:1) and the variable region of the heavy chain (SEQ ID NO:3) of the antibody 225 are as follows, which encode the variable region of the light chain (2L: SEQ ID NO:2) and the variable region of the heavy chain (2H: SEQ ID NO:4), respectively. On the other hand, the base sequence and amino acid sequence of the humanized variable region of the light chain (OL); and the base sequence and amino acid sequence of the humanized variable region of the heavy chain (OH) of the anti-CD3 antibody OKT are disclosed as SEQ ID NO:5 and SEQ ID NO:6 in Patent Document 4; and as SEQ ID NO:7 and SEQ ID NO:8 in Patent Document 5, respectively.

TABLE 1
(SEQ ID NO: 1)
GATATCCAACTGACCCAGTCTCCAGTCATCCTGTCTGTGAGTCCAGGAG
AAAGAGTCAGTTTCTCCTGCAGGGCCAGTCAGAGTATTGGCACAAACAT
ACACTGGTATCAGCAAAGAACAAATGGTTCTCCAAGGCTTCTCATAAAG
TATGCTTCTGAGTCTATCTCTGGGATCCCTTCCAGGTTTAGTGGCAGTG
GATCAGGGACAGATTTTACTCTTAGCATCAACAGTGTGGAGTCTGAAGA
TATTGCAGATTATTACTGTCAACAAAATAATAACTGGCCAACCACGTTC
GGTGCTGGGACCAAGCTGGAGATCAAA

TABLE 2
(SEQ ID NO: 3)
CAGGTACAACTGCAGGAGTCAGGACCTGGCCTAGTGCAGCCCTCACAGA
GCCTGTCCATCACCTGCACAGTCTCTGGTTTCTCATTAACTAACTATGG
TGTACACTGGGTTCGCCAGTCTCCAGGAAAGGGTCTGGAGTGGCTGGGA
GTGATATGGAGTGGTGGAAACACAGACTATAATACACCTTTCACATCCA
GACTGAGCATCAACAAGGACAATTCCAAGAGCCAAGTTTTCTTTAAAAT
GAACAGTCTGCAATCTAATGACACAGCCATATATTACTGTGCCAGAGCC
CTCACCTACTATGATTACGAGTTTGCTTACTGGGGCCAAGGGACCACGG
TCACCGTTTCCTCG

The diabody-type bispecific antibody according to the present invention can take the following four structures in view of the orientation of each variable region.

(1) HL-type: the variable region of the heavy chain is located at an N-end side in each polypeptide.

(2) LH-type: the variable region of the light chain is located at an N-end side in each polypeptide.

(3) O2-type: the variable region of OKT3 is located at an N-end side in each polypeptide.

(4) 2O-type: the variable region of the antibody 225 is located at an N-end side in each polypeptide.

A second aspect of the present invention relates to a tandem-type single chain antibody (scFv) having the structure represented by (2L2H)-(peptide linker)-(OHOL).

The above single chain antibody has the structure of the sixth type disclosed in Patent Document 2. Thus, the single-chain Fv (225 scFv) (5HL) comprising the variable regions of the heavy chain (5H) and the light chain (5L) of the anti-human EGF receptor 1 antibody 225, and the single-chain Fv (OKT3 scFv) (OHL) comprising the humanized variable regions of the heavy chain (OH) and the light chain (OL) of the anti-CD3 antibody OKT3 are linked tandem together via the peptide linker to form a single polypeptide chain as a whole. Either 225 scFv or OKT3 scFv may be positioned at the N-end of the single-chain polypeptide. Furthermore, either heavy chain or light chain may be positioned at the N-end of each scFv. Considering the order of the two kinds of heavy chains and light chains, the sixth type of the present BsAb includes eight kinds of the single-chain polypeptides in total.

As another single chain antibody of the antibody according to the present invention has the structure (E₂₂₅x3 scDb) represented by (OH2L)-(a peptide linker)-(2HOL) of the first type disclosed in Patent Document 2. Thus, the two kinds of the polypeptide chains constituting E₂₂₅x3, OH2L and 2HOL, are further linked together by the peptide linker to form a single polypeptide chain as a whole. As a result, the structure of this BsAb molecule has been more stabilized than E₂₂₅x3. Furthermore, said BsAb may be produced by a single kind of an expression vector, so that more homogeneous BsAb molecule may be prepared than E₂₂₅x3. The term “scDb” means a single-chain diabody-type bispecific antibody.

The above peptide linker may be inserted between 2H and 2L, or between OH and OL. And either VL or VH in each unit of E₂₂₅x3 may be positioned at its N-end. Thus, the first type of the present BsAb comprises each variable region in the order of: (i)N-end:OH-2L-(the peptide linker)-2H-OL:C-end; (ii) N-end:2H-OL-(the peptide linker)-OH-2L:C-end; (iii) N-end:2L-OH-(the peptide linker)-OL-2H:C-end, or (iv) N-end:OL-2H-(the peptide linker)-2L-OH:C-end. The above types (iii) and (iv) correspond to the structure obtained by binding single-chain polypeptides constituting the LH-type diabody-type bispecific antibody.

Furthermore, an antibody may be mentioned as other aspects of the present invention, wherein a hinge region and an Fc region are comprised in addition to the variable region comprising the variable region of the light chain (2L: SEQ ID NO:2) and the variable region of the heavy chain (2H: SEQ ID NO:4) of the anti-human EGF receptor 1 antibody 225, and the humanized variable region of the light chain (OL: SEQ ID NO:6) and the humanized variable region of the heavy chain (OH: SEQ ID NO:8) of the anti-CD3 antibody OKT. The term “Fc region” means a region comprising two domains (CH2 and CH3) constituting a constant region (C region), which is located at C-end side of the heavy chain; and the hinge region.

As examples of the above highly functional bispecific antibody, there may be mentioned the highly functional bispecific antibodies with a conventional IgG-type antibody molecule such as the second (ii) type (Ex3-Fc) and the third (iii) type (Ex3 scDb-Fc) disclosed in Patent Document 2; and the highly functional bispecific antibody that is described as the fourth (iv) type (Ex3 scFv-Fc) comprising a light chain constant region (CL) and a heavy chain constant region (CH1) in addition to the above constituents, wherein “Ex3” is replaced by “E₂₂₅x3.”

As any one of the present BsAb of the types (ii), (iii) and (iv) comprises the human Fc region, it may be easily purified with Protein A. They can further induce an antibody-dependent cellular cytotoxicity (ADCC) and cell-dependent cytokine (CDC). They also show an advantage that they can bind divalently to each antigen, which is not found with E₂₂₅x3.

There is no limitation in the Fc region, the light chain constant region (CO and the heavy chain constant region (CH1) comprised in the bispecific antibody of the fourth type mentioned above as long as they are originated from the human antibody. For example, CL may be originated from κ or λ chain. The CH1 is usually originated from γ chain of IgG. Examples of the CH1, the Fc region and CL are those having an amino acid sequence represented by SEQ ID NO:29, SEQ ID NO:30 and SEQ ID NO:33, respectively, in Patent Document 2. The Fc region of IgG2 type (human IgG2 gene sequence (BX640623.1) of GenBank), which has a lower inducing capability of an effector function, may be used as well.

More specifically, the second type (E₂₂₅x3-Fc) has the structure wherein the diabody-type bispecific antibody (E₂₂₅x3) consisting of the two kinds of the polypeptides of (OH2L) and (2HOL) is bonded to the two Fc regions of the human antibody via each hinge region through either of the two polypeptides. Thus, this is composed of one of the two kinds of the polypeptides constituting E₂₂₅x3 that has been bonded to the Fc region of the human antibody via each hinge region (for example, (2HOL)-(hinge region)-Fc region), and the other polypeptide (for example, OH2L). The above antibody may be produced by expressing the two kinds of the polypeptides and assembling them.

In the antibody of the above type, either 2HOL or OH2L may be bonded to the Fc region of the human antibody via the hinge region, and either the heavy chain variable region or light chain variable region may be bonded to the hinge region.

The third type (E₂₂₅x3 scDb-Fc) has the structure wherein a single-chain polypeptide of (OH2L)-(a peptide linker)-(2HOL) that have been obtained by linking two kids of polypeptides constituting E₂₂₅x3 via a peptide linker (E₂₂₅x3 scDb), a single-chain polypeptide of (OH2H )-(a peptide linker)-(2LOL) or a tandem-type single-chain polypeptide of (2L2H)-(a peptide linker)-(OHOL) is bonded to the Fc region of the human antibody via each hinge region. Any one of the two kinds of the heavy chain variable region or light chain variable region comprised in the single-chain polypeptide may be bonded to the hinge region instead of E₂₂₅x3 in the second type.

As the number of the domains constituting the second and third type of the present antibody is the same as that of an immunoglobulin molecule of the IgG type, it is considered that these antibodies have a steric structure similar to that of the immunoglobulin molecule. A protease cleavage site may be inserted between the hinge region and E₂₂₅x3 or E₂₂₅x3 scDb in the second or third type of the present BsAb. As a result, E₂₂₅x3 or E₂₂₅x3 scDb can be easily produced by digesting these BsAb with the protease followed by the purification steps mentioned below. The E₂₂₅x3 or E₂₂₅x3 scDb thus produced by the protease digestion will show stronger cytotoxic activity than those produced by the conventional methods.

The fourth type (E₂₂₅x3 scFv-Fc) has the structure wherein the VH and VL of the human antibody are replaced by the single-chain Fv (scFv) (2HL) comprising the variable regions of the heavy chain (2H) and the light chain (2L) of the anti-human EGF receptor 1 antibody 225, and the single-chain Fv (OHL) comprising the humanized variable regions of the heavy chain (OH) and the light chain (OL) of an anti-CD3 antibody OKT3, respectively, or vice versa. Thus, this BsAb is an IgG-type immunoglobulin composed of two polypeptides, i.e., a polypeptide wherein one of the scFv of OHL and 2HL is bonded to the N-end of CH1 domain constituting the constant region of the heavy chain, and a polypeptide wherein the other scFv is bonded to the N-end of CL domain constituting the constant region of the light chain. And, either the heavy or light chain variable region in each scFv may be bonded to the constant region. The above antibody may be produced by expressing the two kinds of the single-chain polypeptides and assembling them.

The antibodies according to the present invention further comprise various antibodies wherein the variable region of the light chain is located at the N-end (N-terminal) side of the variable region of the heavy chain in each polypeptide constituting the bispecific antibody (LH-type). Thus, an example of such LH-type of the third type antibody (E₂₂₅x3 scDb-Fc) has the structure wherein the single polypeptide having the structure of (OL2H)-(a peptide linker)-(2LOH) is bonded to the Fc region of the human antibody via the hinge region, as described in an example of the present specification.

Various amino acid mutation and/or replacement may be inserted into the heavy or light chain of the anti-Her 1 antibody 225 in each polypeptide that constitutes the bispecific antibody according to the present antibody, as shown in Patent Document 4 or 5.

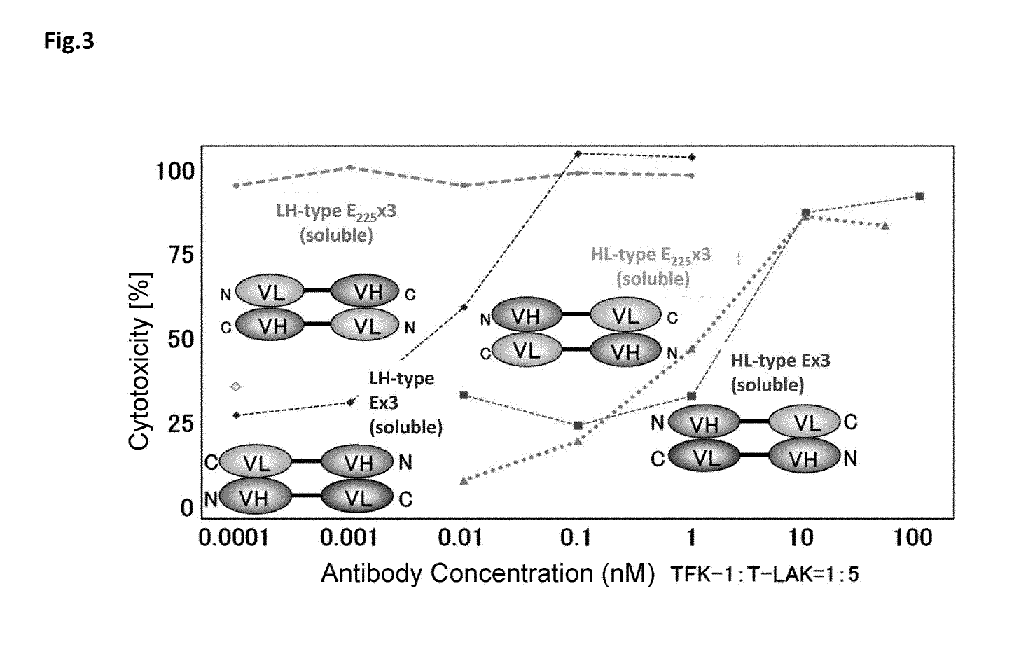

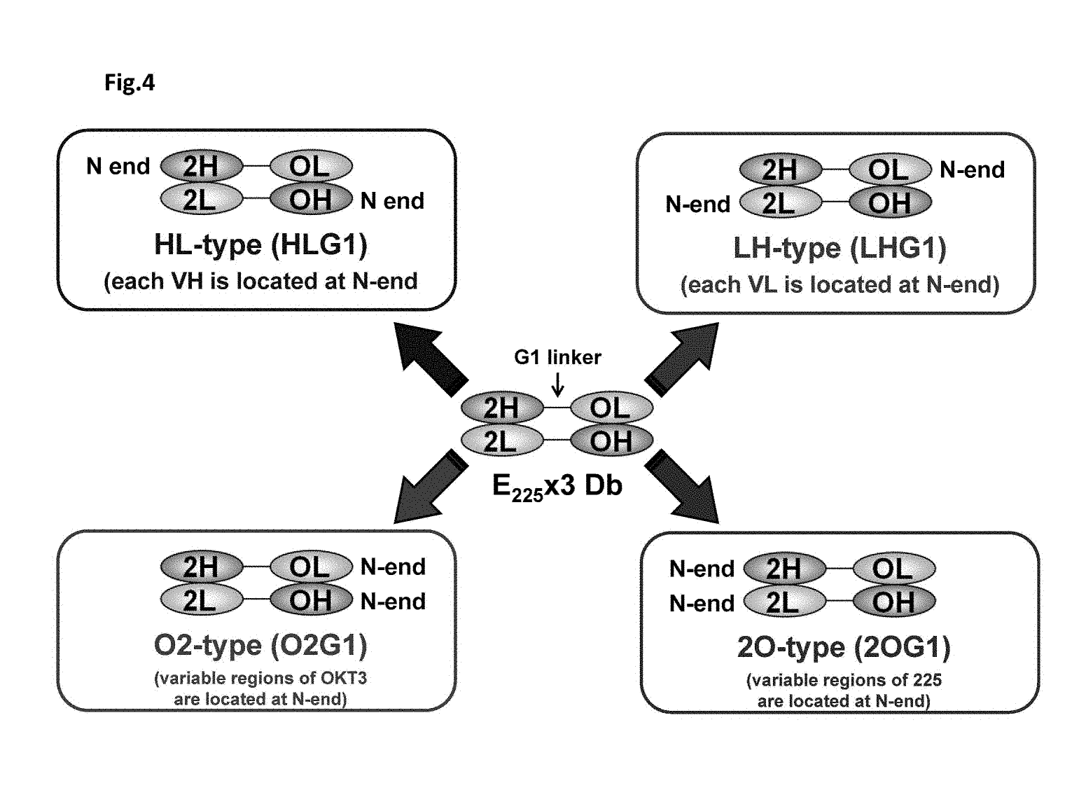

The antibody according to the present invention may comprises amino acid sequences of the PreSission sequence, peptide linker, signal peptide, etc., as shown in FIGS. 3-3 and 3-4 of Patent Document 2. The PreSission sequence comprises a protease-cleavage site. There is no limitation on the kind of protease used in the present invention, so that any enzyme known in the art such as Thrombin and Factor Xa may be used. The amino acid sequence comprising the protease-cleavage site may be optionally selected accordingly.

It is, however, preferable not to comprise the protease-cleavage site in the PreSission sequence in order to more effectively inhibit the fragmentation of the present antibody.

On the other hand, a hybridoma producing the anti-CD3 antibody, OKT3 (ID:TKG0235), is deposited in Cell Resource Center for Biomedical Research, Institute of Development, Aging and Cancer, TOHOKU University, and is also stored at ATCC with an ATCC Accession No. CRL-8001, so that it may be obtained from these deposit authorities.

cDNA may be prepared by known methods using these hybridomas. For example, mRNA is extracted with ISOGEN (Nippon Gene Co.) and then cDNA is prepared by means of First-Strand cDNA Synthesis Kit (Amersham Biosciences Co.). PCR reaction is done for the cDNA using cloning primers that are synthesized in accordance with the disclosure of a Reference document (Krebber, A. et al. Reliable cloning of functional antibody variable domains from hybridomas and spleen cell repertoires employing a reengineered phage display system. J Immunol Methods 201, 35-55. (1997)) so as to determine the sequences of the variable regions of H and L chains of each antibody.

The term “humanized” variable region comprised in the single-polypeptide constituting the bispecific antibody according to the present invention means a human immunoglobulin (a recipient antibody) in which at least a part of the residues of complementary-determining region (CDR) is replaced with residues derived from the CDR of a non-human animal antibody (a donor antibody) that has a desired specificity, affinity and capability, such as those of mouse, rat, and rabbit. In some cases, the residue(s) of a Fv framework (FR) in the human immunoglobulin is replaced with residue(s) of the corresponding non-human antibody. The humanized antibody may further comprise a residue that is not found in the recipient antibody or the introduced CDR or framework. These changes are made in order to optimize or improve the properties of the resulting antibody. More detailed information on these changes are referred to Jones et al., Nature 321, 522-525 (1986); Reichmann et al., Nature 332, 323-329 (1988); EP-B-239400; Presta, Curr. Op. Struct. Biol 2, 593-596 (1992); and EP-B-451216.

The humanized variable region of the antibody may be prepared in accordance with any methods known to those skilled in the art, for example, by analyzing various conceptual humanized preparations based on three-dimensional immunoglobulin models of the recipient antibody and donor antibody, and analyzing them. The three-dimensional immunoglobulin models are well known in the art, being referred to, for example, WO92/22653.

Thus, one example of the humanized variable region according to the present invention is an antibody wherein the complementary determining regions (CDR) in the variable regions are originated from a mouse antibody, and the other parts are originated from a human antibody.

The activity or function of the resulting antibody may be deteriorated due to the humanization. The activity or function of the bispecific antibody according to the present invention may be therefore improved by being provided with a site-specific mutation at an appropriate position in the single-chain polypeptide, for example, at a position in the framework which can affect the CDR structure, such as in canonical sequence or vernier sequence.

It was already reported that the variable region of the humanized OKT3 could sufficiently maintain its activity when compared with the mouse OKT3 (Adair, J. R. et al. Humanization of the murine anti-human CD3 monoclonal antibody OKT3. Hum Antibodies Hybridomas 5, 41-7. (1994)). The total gene was synthesized by means of overlapping PCR based on the amino acid sequence of the variable regions of the humanized OKT3 disclosed in the above document. The optimum codons for the host cell were preferably used in the synthesis. It was also reported that the use of the gene containing the optimum codons would increase the expression level in the host cell.

In addition to the peptide linkers shown in each of the above single-chain polypeptide, it is preferred that the variable regions of the light chain (VL) and the heavy chain (VH) are linked via an appropriate peptide linker. Any linker known in the art or one modified therefrom may be optionally selected and used in the present invention, as long as it makes hard for the single-chain polypeptide to interact within its molecule so that it will enable the formation of a polymer made of plurality of the single-chain antibodies. As a result, the VH and VL comprised in the different single-chain antibodies shall assemble appropriately with each other so as to form a structure that mimics or improves the function of an original protein (the above polypeptide was originated or derived from the original protein) such as all or part of its biological activity. The peptide linker according to the present invention may have 1-20 amino acids, preferably 1-15 amino acids, more preferably 2-10 amino acids.

Alternatively, the two humanized variable regions may be directly linked with each other in the single-chain polypeptide. In such case, one or a few amino acids of the C-end of the variable region located at the N-end side of the single chain polypeptide, or one or a few amino acids of the N-end of the variable region located at the C-end side of the single chain polypeptide are deleted in order to increase three-dimensional degree of freedom in each single-chain antibody and to improve their polymerization.

The polypeptide having an amino acid sequence in which one or a few amino acids such as, for example, 2-5 amino acids are substituted, deleted, inserted or added in the amino acid sequences represented by the above SEQ ID NOS, and having substantially the same property and function as that of the original polypeptide such as an antigen specificity of its variable region may be also used as the single chain polypeptide constituting the present BsAb. it is preferable to make a substitution among amino acids belonging to the same group (polar, non-polar, hydrophobic, hydrophilic, positive-charged, negative-charged, or aromatic amino acid group), or to make a deletion or addition of amino acid so as not to cause a substantial difference or effects with respect to the three-dimensional or local charge-condition of the protein. Such polypeptides having the substitution, deletion or addition of the amino acid(s) my be easily prepared by well known methods such as site-specific mutation (point mutation method or cassette mutation), genetic homologous recombination, primer extension method and PCR, or any optional combinations thereof. The above amino acid sequences comprising one or few amino acids that are substituted, deleted, inserted or added have homology (identity) of 90% or more, preferably 95% or more, more preferably 99% or more with a full-length amino acid sequence of the original amino acid sequence.

The representative examples of the nucleic acid molecules (oligonucleotides) encoding the whole or part of the amino acid sequences of the single-chain polypeptide according to the present invention have the nucleotide sequences shown in the above SEQ ID NOS. Furthermore, as a nucleic acid molecule with the nucleotide sequence having homology of 90% or more, preferably 95% or more, more preferably 99% or more with a full-length nucleotide sequence represented by the above SEQ ID NOS is considered to encode a polypeptide having substantially the same property and function as that of the original polypeptide or part thereof, the above nucleic acid molecule is included in the nucleic acid molecule of the present invention. Although the nucleic acid molecule comprises a nucleotide sequence encoding at least either of the two kinds of the single-chain polypeptides constituting the BsAb according to the present invention, it preferably comprises two kinds of nucleotide sequences together, each one of which encodes one of the two kinds of said single-chain polypeptides, respectively.

In order to determine the homology between two amino acid or nucleotide sequences, they may be preliminarily treated into an optimum condition for comparison. For example, a gap may be inserted into one of the sequences to optimize the alignment with the other sequence, followed by the comparison of amino acid or nucleotide at each site. When the same amino acid or nucleotide exists at a corresponding site of the first and second sequences, these two sequences are considered to be identical with respect to said site. Homology between two sequences is shown by a percent ratio of the number of the identical sites over the total number of amino acids or nucleotides between the two sequences.

The term “homology (identity)” in this specification means an amount (or a number) of the amino acids in an amino acid sequence or the nucleotides in a nucleotide sequence, which are determined to be identical with each other in the relationship between two sequences, showing an extent of the correlation between the two polypeptide or nucleotide sequences. The homology may be easily calculated. The term “homology” or “identity” is well known in the art, and many methods for the calculation of such homology are known, among them. For example, Lesk, A. M. (Ed.), Computational Molecular Biology, Oxford University Press, New York, (1988); Smith, D. W. (Ed.), Biocomputing: Informatics and Genome Projects, Academic Press, New York, (1993); Grifin, A. M. & Grifin, H. G. (Ed.), Computer Analysis of Sequence Data: Part I, Human Press, New Jersey, (1994); von Heinje, G., Sequence Analysis in Molecular Biology, Academic Press, New York, (1987); Gribskov, M. & Devereux, J. (Ed.), Sequence Analysis Primer, M-Stockton Press, New York, (1991). A general method for the determination of the homology between two sequences is disclosed, for example, in Martin, J. Bishop (Ed.), Guide to Huge Computers, Academic Press, San Diego, (1994); Carillo, H. & Lipman, D., SIAM J. Applied Math., 48: 1073 (1988). A preferable method for the determination of the homology between two sequences is, for example, one designed to obtain a largely related part between said two sequences. Some of them are provided as a computer program. Although preferable examples of the computer programs for the determination of the homology between two sequences include, but not limited to, GCG program package (Devereux, J. et al., Nucleic Acids Research, 12(1): 387 (1984)), BLASTP, BLASTN, FASTA (Atschul, S. F. et al., J. Molec. Biol., 215: 403 (1990), any method known in the art may be used.

The nucleic acid of the present invention further includes a DNA molecule that hybridizes with a DNA comprising a nucleotide sequence complementary to the nucleotide sequence represented by the above SEQ ID NOS under stringent conditions, and encodes a polypeptide having substantially the same property and function as that of the polypeptides represented by the above SEQ ID NOS.

Hybridization may be carried out by or in accordance with a method well known in the art such as that described in Molecular cloning third. ed. (cold Spring Harbor Lab. Press, 2001). Hybridization may be done in accordance with an instruction or manual attached to a commercially available library.

Hybridization may be carried out by or in accordance with a method well known in the art such as that described in Current protocols in molecular biology edited by Frederick M. Ausbel et al., 1987). Hybridization may be done in accordance with an instruction or manual attached to a commercially available library.

The phrase “stringent conditions” in this specification may be defined by a suitable combination of salt concentration, organic solvent (for example, formamide), temperature, and other known conditions. Thus, stringency will be increased by the decrease of salt concentration, or the increase of an organic solvent concentration or hybridization temperature. The washing conditions after the hybridization may also affect the stringency. The washing conditions are also defined by salt concentration and temperature. The stringency of washing will be increased by the decrease of salt concentration or the increase of temperature.

Accordingly, the “stringent conditions” in this specification means conditions under which a specific hybrid can be formed only between the nucleotide sequences having homology of about 80% or more, preferably about 90% or more, more preferably about 99% or more on a total average. Specifically, they may be sodium concentration of 150-900 mM, preferably 600-900 mM, pH6-8 at 60-68° C. One example of the stringent conditions is hybridization in 5×SSC (750 mM NaCl, 75 mM Na₃ Citirate), 1% SDS, 5× Denhart solution 50% formaldehyde at 42° C., followed by the washing with 0.1×SSC (15 mM NaCl, 1.5 mM Na₃ Citirate), 0.1% SDS at 55° C.

Furthermore, the nucleic acid encoding the variable regions in the single-chain polypeptide of the present invention may be synthesized by means of the over-lapping PCR method based on a pre-determined amino acid sequence. The nucleic acid used herein has no limitation in its chemical structure or preparation route, as long as it is a molecule encoding the single-chain polypeptide, including gDNA, cDNA chemically-synthesized DNA and mRNA.

Specifically, the nucleic acid according to the present invention may be isolated from cDNA library by means of hybridization or PCR based on the sequences disclosed in literatures. The thus isolated DNA may be inserted in an expression vector, with which a host cell such E. coli, COS cell, CHO cell or myeloma not expressing immunoglobulin are transfected to synthesize a monoclonal antibody in the thus transformed host cells. PCR may be carried out in accordance with a method known in the art, or substantially the same or altered methods. The methods disclosed in, for example, R. Saiki, et al., Science, 230:1350, 1985; R. Saiki, et al., Science, 239:487, 1988; H. A. Erlich ed., PCR Technology, Stockton Press, 1989; D. M. Glover et al., ed., “DNA Cloning,” 2^{nd}. ed., Vol.1, (The Practical Approach Series), IRL Press, Oxford University Press (1995); M. A. Innis et al., ed., “PCR Protocols: a guide to methods and applications,” Academic Press, New York (1990); M. J. McPherson, P. Quirke and G. R. Taylor (Ed.), PCR: a practical approach, IRL Press, Oxford (1991); M. A. Frohman et al., Proc. Natl. Acad. Sci. USA, 85, 8998-9002 (1988), and their modified and altered methods may be used in the present invention. PCR may be performed with use of a commercially available kit in accordance with manufacturer's protocols.

The sequencing method of nucleic acids such as DNA may be referred to Sanger et al., Proc. Natl. Acad. Sci. USA 74:5463-5467 (1977). A general method for recombinant DNA techniques may be referred to J. Sambrook, E. F. Fritsch & T. Maniatis (ed.), “Molecular Cloning: A Laboratory Manual (2^{nd} edition)”, Cold Spring Harbor Laboratory Press, Cold Spring Harbor, N.Y. (1989) and D. M. Glover et al. (ed.), 2^{nd} ed., Vol. 1 to 4 (The Practical Approach Series), IRL Press, Oxford University Press (1995).

The nucleic acid encoding the single-chain polypeptide constituting the present BsAb or each region contained therein may be modified or altered so that it will optionally encode a desired peptide or amino acid depending on the purpose. The techniques for such modification or alternation are disclosed in Mutagenesis: a Practical Approach, M. J. McPherson (ed.), IRL Press, Oxford, UK (1991), including a site-specific mutagenesis introduction method, cassette mutagenesis induction method and PCR mutagenesis method.

The term “modification (or alternation)” as used herein refers to insertion, deletion or substitution of base(s) in at least one codon encoding an amino acid residue in the originally obtained nucleic acid. It includes alternation of the amino acid sequence per se of the single-chain polypeptide by replacing a codon encoding the original amino acid with a codon encoding another amino acid.

Alternatively, the nucleic acid encoding the single-chain polypeptide may be altered without changing the amino acid per se, by using a codon suitable for the host cell such as CHO cell (an optimum codon). With the use of the optimum codon, expression efficiency of the single-chain polypeptide in the host cell will be improved.

The antibody according to the present invention may be produced by various methods well known in the art such as genetic engineering technique and chemical synthesis. For example, the genetic engineering technique includes producing a replicable cloning vector or an expression vector containing the nucleic acid molecule encoding each of the two kinds of the single-chain polypeptides constituting the above bispecific antibody, transforming a host cell with the vector, culturing the transformed host cell to express each of the single-chain polypeptides, collecting and purifying said single-chain polypeptides, assembling the two kinds of the single-chain polypeptides, and separating and collecting the bispecific antibody thus formed.

The term “replicable expression vector” or “expression vector” as used herein refers to a piece of DNA (usually double-stranded) that may comprise a fragment of a foreign DNA fragment inserted therein. The foreign DNA is also defined as a “heterologous DNA”, which can not be found naturally in a host cell in interest. The vector is used to carry or convey the foreign or heterologous DNA into an appropriate host cell. Once the vector is introduced into the host cell, it may be replicated independently from a chromosomal DNA of the host cell to produce copies of the vector and foreign DNA inserted therein. The vector also comprises elements essential for translating the foreign DNA into a polypeptide so that the polypeptide molecules encoded by the foreign DNA will be synthesized very quickly.

The above vector means a DNA construct comprising an appropriate control sequence and DNA sequence that are operably linked together (i.e., linked together so that the foreign DNA can be expressed). The control sequence includes a promoter for transcription, an optional operator sequence to regulate the transcription, a sequence encoding an appropriate mRNA ribosome-biding site, an enhancer, a polyadenylation sequence, and a sequence controlling the termination of transcription and translation. The vector may further comprise various sequences known in the art, such as a restriction enzyme cleaving site, a marker gene (selection gene) such as a drug-resistant gene, a signal sequence, and a leader sequence. These sequences and elements may be optionally selected by those skilled in the art depending on the kinds of the foreign DNA and host cell, and conditions of culture medium. Furthermore, various peptide tags (c-myc and His-tag, for example) known in the art may be contained at its end, etc.

The vector may be in any form such as a plasmid, phage particle, or just simply genomic insert. Once the appropriate host cell is transformed with the vector, the vector will be replicated or function independently from the genome of the host cell, or the vector will alternatively be integrated into the genome of the cell.

The various expression vectors that are used in the production of the single polypeptide constituting the antibody according to the present invention may be easily constructed by those skilled in the art using the techniques known in the art. Examples of the above vectors are described in Patent Document 1, especially in Examples 1, 2, 11 and 12, and examples of Patent Document 2.

Any cell known in the art may be used as the host cell, for example, there may be mentioned prokaryotic cells such as including E. coli, eukaryotic cells such as mammalian cells such Chinese hamster ovary (CHO) cell and human cells, yeast, and insect cells. For example, BL21 star (DE3) strain is cultured in 2×YT culture medium at about 28° C. and induced with IPTG of about 0.5 mM, so that the yield of the present LK-type bispecific antibody may be highly improved so as to increase its production efficiency.

Although the single-chain polypeptide obtained by the expression in the host cell is usually secreted and collected from the culture medium, it may be also collected from cell lysate when it is directly expressed without a secretion signal. In case the single-chain polypeptide has a membrane-binding property, it may be released from the membrane with an appropriate surfactant such as Triton-X100.

Purification of the polypeptide may be carried out by any method known to those skilled in the art such as centrifugation, hydroxyapatite chromatography, gel electrophoresis, dialysis, separation on ion-exchange chromatography, ethanol precipitation, reverse phase HPLC, silica chromatography, heparin-sepharose chromatography, anion- or cation-resin chromatography such as polyaspartic acid column, chromato-focusing, SDS-PAGE, precipitation with ammonium sulfate, and affinity chromatography. The affinity chromatography, which utilizes affinity with a peptide tag of the single-chain polypeptide, is one of the preferred purification techniques with a high efficiency.

Since the collected single-chain polypeptide may be often included in an insoluble fraction, the polypeptide is preferably purified after being solubilized and denatured. The solubilization treatment may be carried out with the use of any agent known in the art, including alcohol such ethanol, a dissolving agent such as guanidine hydrochloride and urea. The present BsAb is produced by assembling or rewinding the two kinds of the single-chain polypeptides thus purified, and separating and collecting the thus formed antibody molecule.

Assembling treatment will bring a single-chain polypeptide back in its appropriate spatial arrangement in which a desired biological activity is shown. Since this treatment may also bring polypeptides or domains back into their assembling state, it may be considered “re-assembling.” It may be also called “re-constitution” or “refolding” in view of gaining the desired biological activity. The assembling treatment may be carried out by any method known in the art, preferably by gradually lowering the concentration of a denaturing agent such as guanidine hydrochloride in a solution comprising the single-chain polypeptide by means of dialysis. During these processes, an anti-coagulant or oxidizing agent may be optionally added in a reaction system in order to promote the oxidation. The separation and collection of the polymerized low-molecular antibodies thus formed may be done by any method known in the art as well.

As already described above, the antibody according to the present invention may be prepared from the supernatant of a culture medium, periplasm fraction, intracellular soluble fraction and intracellular insoluble fraction.

It is possible to transform the host cell with the co-expression vector containing the nucleic acid molecule encoding each of the single-chain polypeptides constituting the antibody of the present invention, or with the two kinds of the expression vector containing the nucleic acid molecule encoding each of said single-chain polypeptides, respectively, culturing the transformed host cell so as to express each of the single-chain polypeptides, allowing the transformed cell to form the antibody in said cell, and separating and collecting it from supernatant of the culture medium or intracellular soluble fraction. In such case, the above assembling or rewinding treatment is unnecessary so that a high productivity can be achieved at a low cost.

A pharmaceutical composition according to the present invention comprises an active ingredient selected from the group consisting of the antibody according to the present invention, the single-chain polypeptide, the nucleic acid, the vector, and the host cell described in the above. As shown by the examples in the present specification, since the active ingredient has an activity of eliminating, hurting, damaging and/or reducing tumor cells expressing EGFR in vitro and in vivo, the present pharmaceutical composition is used as an anti-tumor agent.

An effective amount of the active ingredient may be optionally determined by those skilled in the art depending on the purpose of treatment, medical conditions of a patient to be treated such as kind, site or size of tumor, and administration route. A typical dose or daily dose may be first determined in vitro by using an assay method of growth or existence of the tumors known in the art, then determined with use of such an appropriate animal model as to allow extrapolation of the resulting dose range to human patients.

The pharmaceutical composition of the present invention may optionally comprise various kinds of pharmaceutically acceptable components known in the art such as carrier, excipient, buffer, stabilizing agent and the like, depending on various factors such as the kind of the active ingredients, its formulation form, the route and purpose of administration, medical conditions of patient.

The pharmaceutical composition of the present invention may be formulated into any form such as pill, liquid, powder, gel, air spray, microcapsule, and colloidal dispersion (liposome, micro emulsion, etc.).

The pharmaceutical preparation may be administered by injecting or infusing intraveneously, intraperitoneally, intracerebrally, intraspinally, intramuscularly, intraocularly, intraarterially, especially intrabiriarily, or via diseased tissue, or with use of a constant releasing agent system. The active ingredient according to the present invention may be administered through continuous fluid infusion or massive injection. The pharmaceutical composition according to the present invention is preferably administered in combination with the cell having phagocytosis or cytotoxic activity. Alternatively, the active ingredient such as the present LH-type diabody-type BsAb may be mixed with the above cells so as to bind to them before its administration.

The constant releasing agent generally refers to a formulation that can release the active ingredient of the present invention for a certain period of time. One of the preferred constant releasing agents comprises a semi-permeable carrier of solid hydrophobic polymer such as protein, which is shaped into a form such as film or micro capsule.

The pharmaceutical preparation according to the present invention may be produced by a method that is optionally selected from, for example, “Guide Book of Japanese Pharmacopoeia”, Ed. of Editorial Committee of Japanese Pharmacopoeia, Version No. 13, published Jul. 10, 1996 by Hirokawa publishing company

The terms as used in the present specification and drawings are based on IUPAC-IUB Commission on Biochemical Nomenclature or on meanings of the terms conventionally used in the art.

The present invention will be explained more in detail by referring to the Examples, which are provided only for describing the specific embodiments of the present invention, but not for limiting the scope of the present invention. It is therefore to be understood that various embodiments based on the inventive concept of the present specification may be practiced within the scope of the present invention.

The following examples were or can be carried out with standard techniques well known to those skilled in the art unless otherwise described. Thus, unless otherwise described, specific procedures and treating conditions are in accordance with J. Sambrook, E. F. Fritsch & T. Maniatis, “Molecular Cloning”, 2nd ed., Cold Spring Harbor Laboratory, Cold Spring Harbor, N.Y. (1989) and D. M. Glover et al. ed., “DNA Cloning”, 2nd ed., Vol. 1 to 4, (The Practical Approach Series), IRL Press, Oxford University Press (1995) (DNA cloning), and with H. A. Erlich ed., PCR Technology, Stockton Press, 1989 ; D. M. Glover et al. ed., “DNA Cloning”, 2nd ed., Vol. 1, (The Practical Approach Series), IRL Press, Oxford University Press (1995) and M. A. Innis et al. ed., “PCR Protocols”, Academic Press, New York (1990) (PCR). A commercially available agent and kit were used in accordance with protocols attached thereto.

### EXAMPLE 1

### Construction of E₂₂₅x3 Expression Vectors (HL-type, LH-type, Tandem scFv-type)

### Production of pRA1-2HOL and pRA1-OH2L

An expression vector was constructed as follows based on the expression vector, pRA-5HOL and pRA-OH5L for the humanized diabody-type bispecific antibody for EGFR and CD3 (Patent Document 1). pRA1-2HOL was constructed by amplifying 2H with PCR using primers represented by SEQ ID NO:9 and NO:10, digesting with NcoI and EagI and replacing the 5H part in pRA-SHOL with 2H. pRA1-OH2L was then constructed by amplifying 2L with PCR using primers represented by SEQ ID NO:11 and NO:12, digesting with EcoRV and SacII and replacing the 5L part in pRA-OH5L with 2L.

back primer (NcoI-225H)
(SEQ ID NO: 9)
5′-NNNCCATGGCCCAGGTACAACTGCAGGAGTCAGGACC
TGGCCTAGTGCAGC-3′
forward primer (225H-EagI)
(SEQ ID NO: 10)
5′-NNNCGGCCGAGGAAACGGTGACCGTGGTCCCTTGGCC
CCAGTAAGC-3′
back primer (EcoRV-225L)
(SEQ ID NO: 11)
5′-NNNGATATCCAACTGACCCAGTCT-3′
forward primer (225L-SacII)
(SEQ ID NO: 12)
5′-NNNCCGCGGCACGTTTGATCTCCAGCTTGGTCCC-3′

(1) Construction of HL-type E₂₂₅x3 Db Co-expression Vector

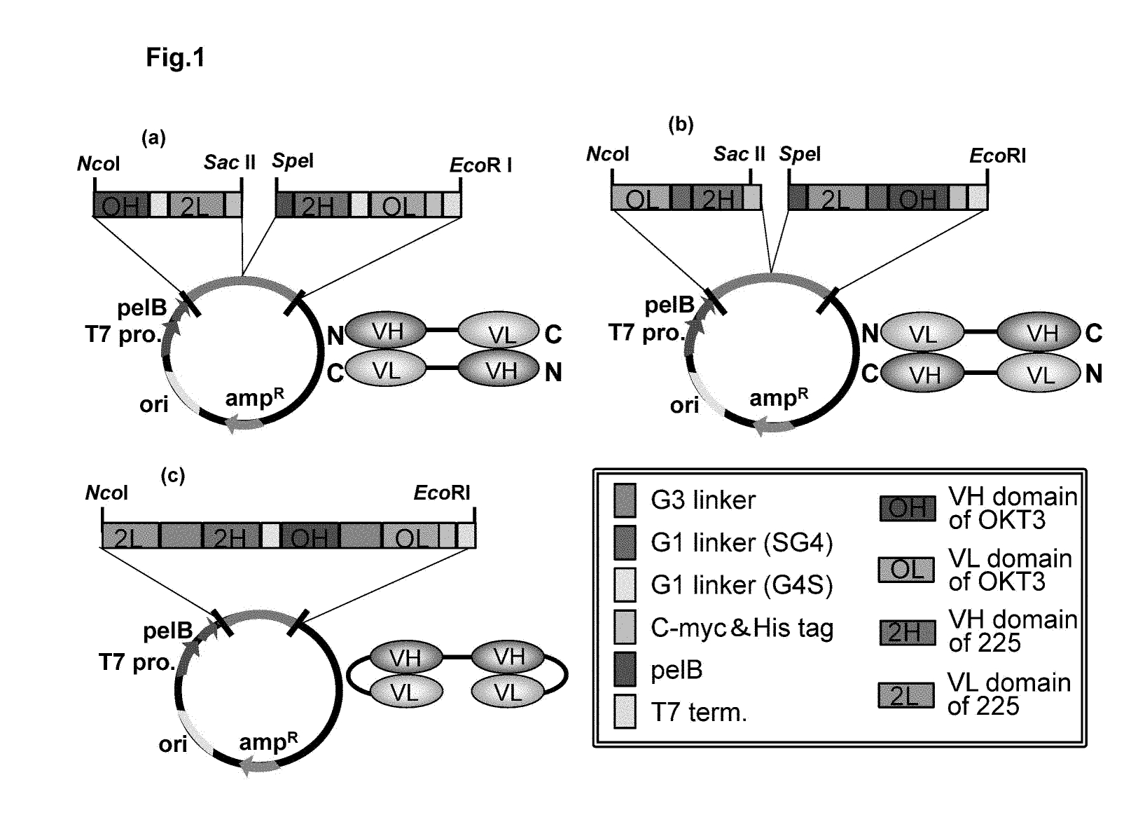

pRA1-2HOL was subjected to PCR using the following primers, and the resulting PCR product was digested with SpeI and EcoRI, and ligated to give the HL-type co-expression vector, pRA1-E₂₂₅x3 HLG1 (FIG. 1a).

<<Primers for the construction of pRA1-E₂₂₅x3 HLG1>>
back primer (SpeI-pelB)
(SEQ ID NO: 13)
5′-NNNACTAGTTATTTCAAGGAGACAGTCATAATGAAATAC-3′
forward primer (T7term-EcoRI)
(SEQ ID NO: 14)
5′-NNNGAATTCATCCGGATATAGTTCCTCCTTTCAG-3′

(2) Construction of LH-type E₂₂₅x3 Db Co-expression Vector

2H was amplified with PCR using the primers of SEQ ID NO:9 and NO:15, and the resulting PCR product was digested with Ncol and EcoRV. The OH part in pRA1-OH2L was replaced with the resulting product by means of ligation reaction to obtain pRA1-2H2L.

back primer (NcoI-225H)
(SEQ ID NO: 9)
5′-NNNCCATGGCCCAGGTACAACTGCAGGAGTCAGGACCTGGCCTA
GTGCAGC-3′
forward primer (225H-G3-EcoRV)
(SEQ ID NO: 15)
5′-NNNGATATCGGATCCGCCACCGCCCGACCCGCCACCGCCGCTAC
CGCCCCCGCCGGCCGAGGAAACGGTGACCGTGG-3′

First, NcoI-2L-G1-OH, 2L-G1-OH-SacII and NcoI-OL-G1-2H, and OL-G1-2H-SacII were prepared by 1^{st} PCR using said pRA1-2H2L and pRA1-5LOH (the same meaning as “pRA-h5LhOL”) as a template. NcoI-2L-G1-OH-SacII and NcoI-OL-G1-2H-SacII were then prepared by 2^{nd} PCR using the above products as a template. The amplified products were digested with Ncol and SacII, and ligated to pRA1 vector already digested in the same way to give pRA1-2LOH and pRA1-OL2H. The LH-type co-expression vector, pRA1-E₂₂₅x3 LHG1 (FIG. 1b) was then constructed in the way as in the above HL-type vector.

<<Primers for the Construction of pRA1-E₂₂₅x3 LHG1>>

1^{st} PCR

back primer (NcoI-2L)
(SEQ ID NO: 16)
5′-NNNCCATGGCCGATATCCAACTGACCCAGTCTCCAG
TCATCCT-3′
forward primer (2L-G1-OH)
(SEQ ID NO: 17)
5′-ACCTGGCCACCGCCACCAGATTTGATCTCCAGCTTG
GTCCCAGCACCGAA-3′
back primer (2L-G1-OH)
(SEQ ID NO: 18)
5′-AGATCAAATCTGGTGGCGGTGGCCAGGTGCAACTGG
TGCA-3′
forward primer (OH-SacII)
(SEQ ID NO: 19)
5′-NNNNAGCCGCGGAGCTAACGGTCACCGGGGTGCCCT
GGCC-3′

2^{nd} PCR

back primer (NcoI-2L)

forward primer (OH-SacII)

The sequences were described above

<<Primers for the Construction of pRA1-OL2H>>

1^{st} PCR

back primer (NcoI-OL)
(SEQ ID NO: 20)
5′-NNNNCCATGGCCGATATTCAGATGACCCAGAGCCCG-3′
forward primer (OL-G1-2H)
(SEQ ID NO: 21)
5′-TTGTACCTGGCCACCGCCACCAGAGGTAATCTGCAGTTT
GG-3′
back primer (OL-G1-2H)
(SEQ ID NO: 22)
5′-ATTACCTCTGGTGGCGGTGGCCAGGTACAACTGCAGGAG
TCAGGACCT-3′
forward primer (2H-SacII)
(SEQ ID NO: 23)
5′-NNNNCCGCGGAGGAAACGGTGACCGTGGTCC-3′

2^{nd} PCR

back primer (NcoI-OL)

forward primer (2H-SacII)

The sequences were described above

(3) Construction of Tandem scFv-type E₂₂₅x3 Expression Vector

Construction of pRA1-5L5HOHOL

pRA1-5L5HOHOL was constructed by amplifying Ex3 tandem scFv expression vector, pKHI-Ex3 tandem scFv (Patent Document 2) as a template with PCR using primers represented by SEQ ID NO:24 and NO:25, digesting with NcoI and SacII and replacing the OH5L part in pRA-OH5L with the resulting product by means of ligation reaction.

back primer (NcoI-5L)
(SEQ ID NO: 24)
5′-NNNNCCATGGCCGATATTGTGATGACCCAGAGCCCG-3′
forward primer (OL-SacII)
(SEQ ID NO: 25)
5′-NNNNAGCCGCGGCGCGGGTAATCTGCAGTTTGGTACC-3′

First, NcoI-2L-EcoRI and 2L-G3-2H-SacII were prepared by 1st PCR using said pRA1-2H2L as a template. NcoI-2L-G3-2H-SacII was prepared by 2nd PCR using the above products as a template. The amplified products were digested with NcoI and SacII, and ligated to pRA1 vector already digested in the same way to give pRA1-2L2H. Then, 1^{st} PCR was carried out using pRA1-2L2H and pRA1-5L5HOHOL as a template and the following primers to give NcoI-2L-G3-2H and 2H-G1-OH-OL-SacII. NcoI-2L-G3-2H-G1-OH-OL-SacII was prepared by 2nd PCR using the above products as a template. The amplified products were digested with NcoI and SacII, and ligated to pRA1 vector already digested in the same way to give pRA1-2L2HOHOL (FIG. 1c).

<<Primers for the Construction of pRA1-2L2H>>

1^{st} PCR

back primer (NcoI-2L)
(SEQ ID NO: 16)
5′-NNNCCATGGCCGATATCCAACTGACCCAGTCTCCAGTCATCCT-3′
forward primer (T7term-EcoRI)
(SEQ ID NO: 14)
5′-NNNGAATTCATCCGGATATAGTTCCTCCTTTCAG-3′
back primer (2L-G3-2H)
(SEQ ID NO: 26)
5′-AAAGGCGGGGGCGGTAGCGGCGGTGGCGGGTCGGGCGGTGGCGGAT
CCCAGGTACAACTGCAGGAGTC-3′
forward primer (2H-SacII)
(SEQ ID NO: 23)
5′-NNNNCCGCGGAGGAAACGGTGACCGTGGTCC-3′

2^{nd} PCR

back primer (NcoI-2L)

forward primer (2H-SacII)_{°}

The sequences were described above

<<Primers for the Construction of pRA1-2L2HOHOL>>

1^{st} PCR

back primer (NcoI-2L)
(SEQ ID NO: 16)
5′-NNNCCATGGCCGATATCCAACTGACCCAGTCTCCAGTCATCCT-3′
forward primer (2L-G3-2H)
(SEQ ID NO: 27)
5′-CTGGGATCCGCCACCGCCCGACCCGCCACCGCCGCTACCGCCCCCG
CCTTTGATCTCCAGCTTGGTCC-3′
back primer (2H-G1-OH)
(SEQ ID NO: 28)
5′-GTTTCCTCCGGCGGGGGCGGTTCGCAGGTGCAA-3′
forward primer (OL-SacII)
(SEQ ID NO: 25)
5′-NNNNAGCCGCGGCGCGGGTAATCTGCAGTTTGGTACC-3′

2^{nd} PCR

Back primer (NcoI-2L)

Forward primer (OL-SacII)

The sequences were described above

### EXAMPLE 2

### Preparation of E₂₂₅x3 (HL-type, LH-type, tandem scFv-type)

A commercially available competent cell, BL21 (DE3) and BL21(DE3)star (Life technologies Japan Co. Ltd.) were transformed with the vectors constructed in Example 1, cultured in a test tube scale (LB culture medium, 3 mL) at 28° C. and subjected to SDS-PAGE and Western blotting to confirm their expression. As dense bands were observed in BL21(DE3) star, it was determined that the culture would be carried out using this culture medium.

In the culture using BL21(DE3) star, the expression of HL-type and LH-type E₂₂₅x3 was confirmed most clearly under the conditions of 28° C.→20° C. (O.D.=0.8), and was also observed from a soluble fraction. The expression of tandem scFv-type E₂₂₅x3 was confirmed most clearly under the conditions of “staying at 28° C. ”, but was hardly observed from a soluble fraction. It was therefore determined that the mass production would be done in these conditions.

After the culture in the above conditions, the culture medium was subjected to centrifugation to collect supernatant as a soluble fraction. The precipitate was subjected to Osmotic Shock treatment, and centrifuged to collect soluble proteins present in a periplasm fraction as a soluble fraction together with the supernatant (Soluble Fraction (1)). The resulting precipitate was then solubilized with BugBuster reagent, centrifuged and divided into supernatant (Soluble Fraction (2)) and precipitate (Insoluble Fraction).

The expression of each fraction was confirmed by means of SDS-PAGE and Western blotting known for those skilled in the art. As a result, the expression of the HL-type was confirmed in the fractions except the soluble fraction obtained after the treatment with BugBuster reagent, and the expression of the LH-type was confirmed in all of the fractions. The expression of the tandem scFv-type was confirmed only in the soluble fraction after ultrasonic disruption treatment and insoluble fraction.

### Preparation from Soluble Fraction by means of Ammonium Sulfate Precipitation

Ammonium sulfate was gradually dissolved in the above Soluble Fraction (1) to a final amount of 60% mass, stirred overnight at a low room temperature and centrifuged to collect a precipitate. The resulting precipitate was then dissolved into PBS. The resulting sample was purified by means of metal-chelate affinity chromatography:IMAC. Each purification degree was confirmed with the SDS-PAGE and Western blotting. Elution Fraction 3 was purified for the HL-type and LH-type, and Elution Fraction 2 for the tandem scFv-type were purified by means of gel filtration chromatography, respectively. The results showed that an aimed band was clearly confirmed for the HL-type, so that the HL-type E₂₂₅x3 with a high purity was obtained in an amount of 25 μg/L based on absorbance. No aimed band was confirmed for the LH-type or tandem scFv-type.

TABLE 3
After Ni-Sepharose Purification

M: Markers
1: Pass through Fraction
2: Washing Fraction (PBS)
3: Elution Fraction 1 (50 mM)
4: Elution Fraction 2 (150 mM)
5: Elution Fraction 3 (300 mM \textcircled{1})
6: Elution Fraction 4 (300 mM \textcircled{2})
7: Elution Fraction 5 (1 M \textcircled{1})
C: Control (HLG1)
Eluting solution: Imidazole/PBS (pH 8.0)

### Preparation From Soluble Fraction by Means of Cross-Flow

Since there were too much contaminant in the LH-type obtained from the soluble fraction by means of ammonium sulfate precipitation, Soluble Fraction (1) was condensed by means of cross-flow. The tandem-type was not subjected to the purification since no expression was confirmed. The condensed sample was purified by means of IMAC under the conditions listed in Table 3 above and confirmed with the SDS-PAGE and Western blotting, so that Elution Fraction 4 was purified for the HL-type and LH-type by means of gel filtration chromatography. As a result, the HL-type could not be purified due to too much contaminant. The LH-type could be purified in an amount of 96 μg/L based on absorbance with a little amount of contaminant.

### Preparation From Soluble Fraction After the Treatment With BugBuster Reagent

Soluble Fraction (2) was dialyzed against PBS and purified by means of IMAC under the conditions of Table 3. Each purification degree was confirmed with the SDS-PAGE and Western blotting. Like the preparation from the soluble fraction by means of ammonium sulfate precipitation, Elution Fraction 3 was purified for the HL-type and LH-type, and Elution Fraction 2 was purified for the tandem scFv-type by means of gel filtration chromatography, respectively. As a result, all of the HL-type, LH-type and tandem scFv-type could not be purified due to too much contaminant compared to an aimed band.

### Preparation from Insoluble Fraction by Means of Rewinding Method

Insoluble Fraction was solubilized with 6M guanidine HCl aqueous solution (PBS) and purified by means of IMAC under the following conditions. Purification degree of each sample was confirmed with the SDS-PAGE and Western blotting, so that an aimed protein could be purified in Elution Fraction 4 (300 mM) for all of the HL-type, LH-type and tandem scFv-type. This fraction was put into a dialysis membrane, and subjected to rewinding operation by lowering the concentration of an outer guanidine HCl aqueous solution from 6M to 3M, 2M, 1M by every 6 hours, and 0.5M, 0M by every 12 hours (400 mM L-arginine was added as anti-coagulant under 1M, 0.5M and 0M of guanidine HCl aqueous solution) and finally removing L-arginine. Solubilizing ratios were 16%, 12% and 6% for the HL-type, LH-type and tandem scFv-type, respectively. Inner solution after the rewinding was centrifuged, and after the aggregated proteins were removed, the resulting supernatant was purified by means of gel filtration chromatography. The results showed that an aimed band was clearly confirmed for all of the HL-type, LH-type and tandem scFv-type, so that the E₂₂₅x3 with a high purity was obtained in an amount of 340 μg/L, 192 μg/L and 126 μg/L based on absorbance of the HL-type, LH-type and tandem scFv-type, respectively.

TABLE 4
After Ni-Sepharose Purification

M: Markers
1: Pass through Fraction
2: Washing Fraction (PBS)
3: Elution Fraction 1 (20 mM \textcircled{1})
4: Elution Fraction 2 (20 mM \textcircled{2})
5: Elution Fraction 3 (20 mM \textcircled{3})
6: Elution Fraction 4 (300 mM)
7: Elution Fraction 5 (1 M)
C: Control (HLG1)
Eluting solution: Imidazole/PBS (pH 8.0)

### EXAMPLE 3

### Cytotoxicity Test of the Antibodies of the Present Invention

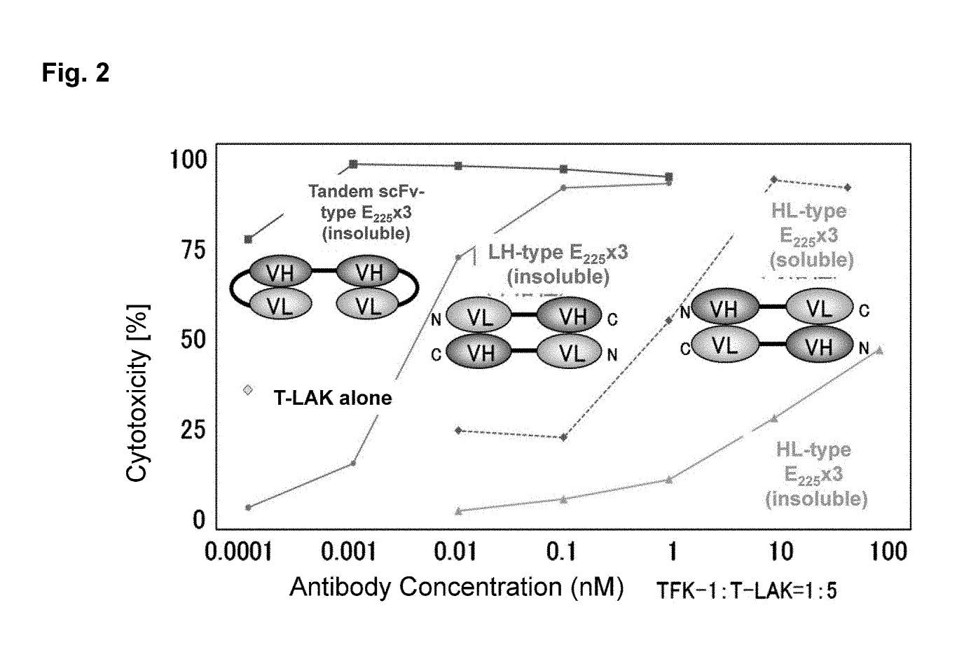

Cytotoxicity test was carried out with respect to four samples of the HL-type, LH-type and tandem scFv-type E₂₂₅x3 prepared from the Insoluble Fraction, and the HL-type E₂₂₅x3 prepared from Soluble Fraction by means of ammonium sulfate precipitation. As shown in Table 5, MTS assay for an EGFR-positive human cholangioma cell strain, TFK-1 was carried out using T-LAK as an effector cell. The results are shown in FIG. 2. TFK-1 has been deposited with Cell Resource Center for Biomedical Research, Institute of Development, Aging and Cancer, TOHOKU University, ID:TKG036.

The results showed that cytotoxicity of the tandem scFv-type was higher than that of the LH-type, which was higher than that of the HL-type, with respect to E₂₂₅x3 prepared from Insoluble Fraction. Considering the fact that the tandem scFv-type was almost the same cytotoxicity as that of the LH-type, which is higher than that of the HL-type with respect to Ex3, it has been revealed that cytotoxicity of the tandem scFv-type and the LH-type is higher than that of the HL-type in both cases. It was also revealed that cytotoxicity of the HL-type E₂₂₅x3 prepared from Soluble Fraction was higher than that prepared from Insoluble Fraction. Considering the fact that there was no substantial difference in cytotoxicity between the HL-type Ex3 prepared from Soluble Fraction and that prepared from Insoluble Fraction, it is suggested that a correct refolding in rewinding is more difficult for E₂₂₅x3 molecule than for Ex3.

Accordingly, the HL-type E₂₂₅x3 and the LH-type E₂₂₅x3 prepared from Soluble Fraction were compared with the HL-type Ex3 and the LH-type Ex3 with respect to cytotoxicity. A sample prepared by means of ammonium sulfate precipitation and cross-flow was used for the HL-type E₂₂₅x3 and the LH-type E₂₂₅x3, respectively. The results are shown in FIG. 3.

FIG. 3 shows that the cytotoxicity of the LH-type was higher than that of the HL-type with respect to both E₂₂₅x3 and Ex3. However, while the cytotoxicity of the HL-type was almost the same with respect to both E₂₂₅x3 and Ex3, the cytotoxicity of the LH-type E₂₂₅x3 was higher than that of LH-type Ex3. These results suggested that while the cytotoxicity of the LH-type was higher than that of the HL-type in low-molecular bispecific antibodies, the degree of the difference in the cytotoxicity would be different in each antibody.

### EXAMPLE 4

### Preparation of Four Kinds of E₂₂₅x3 Db Having Different Orientation

Four kinds of antibodies having different orientation were prepared and subjected to function analysis with respect to the E₂₂₅x3 Dbs according to the present invention. Among the four kinds of orientation of the E₂₂₅x3 Db (FIG. 4), the co-expression vectors were constructed based on the vector described in Example 1 for the O2-type (O2G1) wherein the domains derived from OKT3 was located at an N-end (N-terminal) side and for the 2O-type (2OG1) wherein the domains derived from the 225 antibody was located at an N-end (N-terminal) side.

### Construction of Co-expression Vector of E₂₂₅x3 O2G1

For the construction of co-expression vector of E₂₂₅x3 O2G1, PCR was carried out using pRA1-OL2H as a template under the conditions. Since BamHI site (GGATCC) was present in the 2L gene sequence, the vector would be constructed without using BamHI.

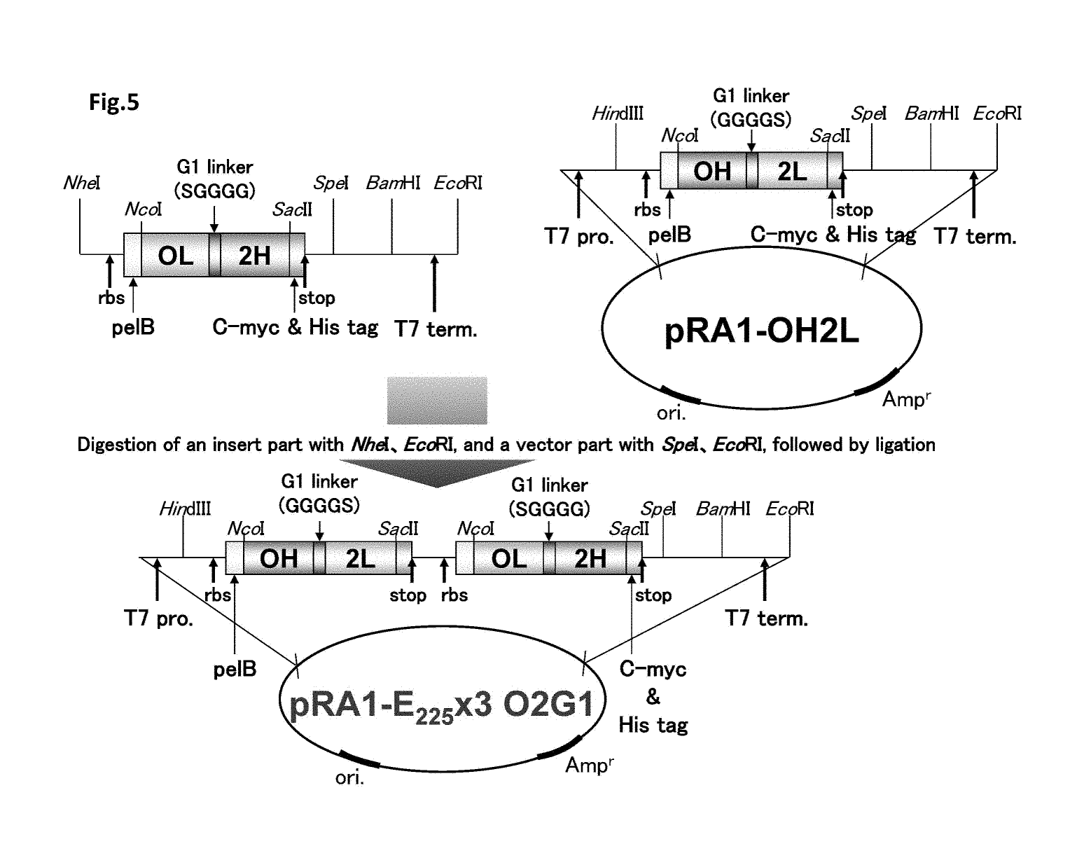

The OL2H fragment (insert part) amplified by the above PCR were digested with restriction enzymes NheI and EcoRI, and pRA1-OH2L (vector part) stored in our laboratory were digested with restriction enzymes SpeI and EcoRI. The resulting fragments were ligated to yield pRA1-E₂₂₅x3 O2G1 (FIG. 5). As both the NheI digestion and the SpeI digestion produced the same cohesive end (CTAG), the above fragments could be ligated with each other.

### Construction of E₂₂₅x3 2OG1 Co-expression Vector

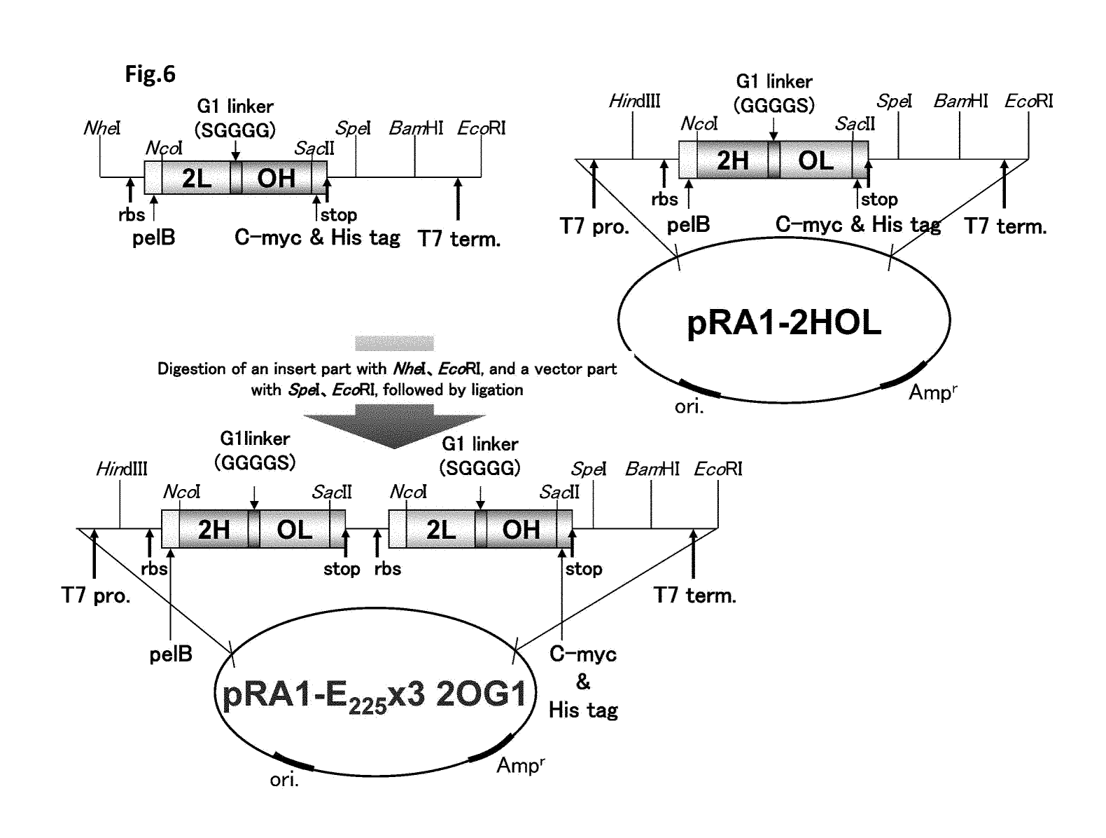

A 2LOH fragment (insert part) amplified with PCR under the same conditions of the construction of co-expression vector of E₂₂₅x3 O2G1 was digested with NheI and EcoRI, and inserted into the NheI and EcoRI site of pRA1-2HOL(vector part) to give E₂₂₅x3 2OG1 co-expression vector (FIG. 6).

### Preparation of Four Kinds of E₂₂₅x3 Db

BL21(DE3) star was transformed with each of the co-expression vectors, pRA1-E₂₂₅x3 HLG1 and pRA1-E₂₂₅x3 LHG1 (Example 1, FIG. 1), and pRA1-E₂₂₅x3 O2G1 and pRA1-E₂₂₅x3 2OG1. The supernatant fraction of the culture medium and the periplasm fraction were combined and condensed with cross-flow and dialyzed according to Example 2. The mass culture was done in 2L and 4L of 2×YT medium for E₂₂₅x3 HLG1 and E₂₂₅x3 LHG1, respectively, and in 3L of the same medium for E₂₂₅x3 O2G1 and E₂₂₅x3 2OG1. The detailed procedures are described below.

The confirmation of the expression with the SDS-PAGE and Western blotting showed that an aimed protein was expressed in the soluble fraction for E₂₂₅x3 HLG1, E₂₂₅x3 LHG1 and E₂₂₅x3 2OG1, but substantially no expression was observed for E₂₂₅x3 O2G1. It may be because that E₂₂₅x3 O2G1 was a molecule unlikely to be secreted into the soluble fraction compared to the other orientations, or a tag for the detection could be cut or any human error might occur during the preparation a sample for SDS-PAGE. However, in order to compare the yields of the four kinds of E₂₂₅x3 Dbs having different orientation. E₂₂₅x3 O2G1 was prepared from the supernatant fraction of the culture medium and the periplasm fraction as well.

The results of condensation with cross-flow and dialysis followed by IMAC purification showed that although the largest amount of the aimed protein was observed in an elution fraction eluted with 300 mM imidazole, a lot of contaminant proteins were also mixed unlike the case of Ex3. The results with Western blotting also revealed that E₂₂₅x3 2OG1 molecule was susceptible to degradation. In order to increase the purity of the aimed protein, the 300 mM imidazole elution fraction was collected, dialyzed against PBS and subjected again to IMAC purification.

As a result, since a fraction with a relatively high purity could be obtained for E₂₂₅x3 HLG1 and E₂₂₅x3 LHG1, the 300 mM imidazole elution fraction was further purified by means of gel filtration chromatography. On the other hand, as the contaminant proteins with a molecular weight similar to that of the aimed protein could not be removed from E₂₂₅x3 2OG1 and E₂₂₅x3 O2G1, the 300 mM imidazole elution fraction was collected, dialyzed and subjected again to IMAC purification.

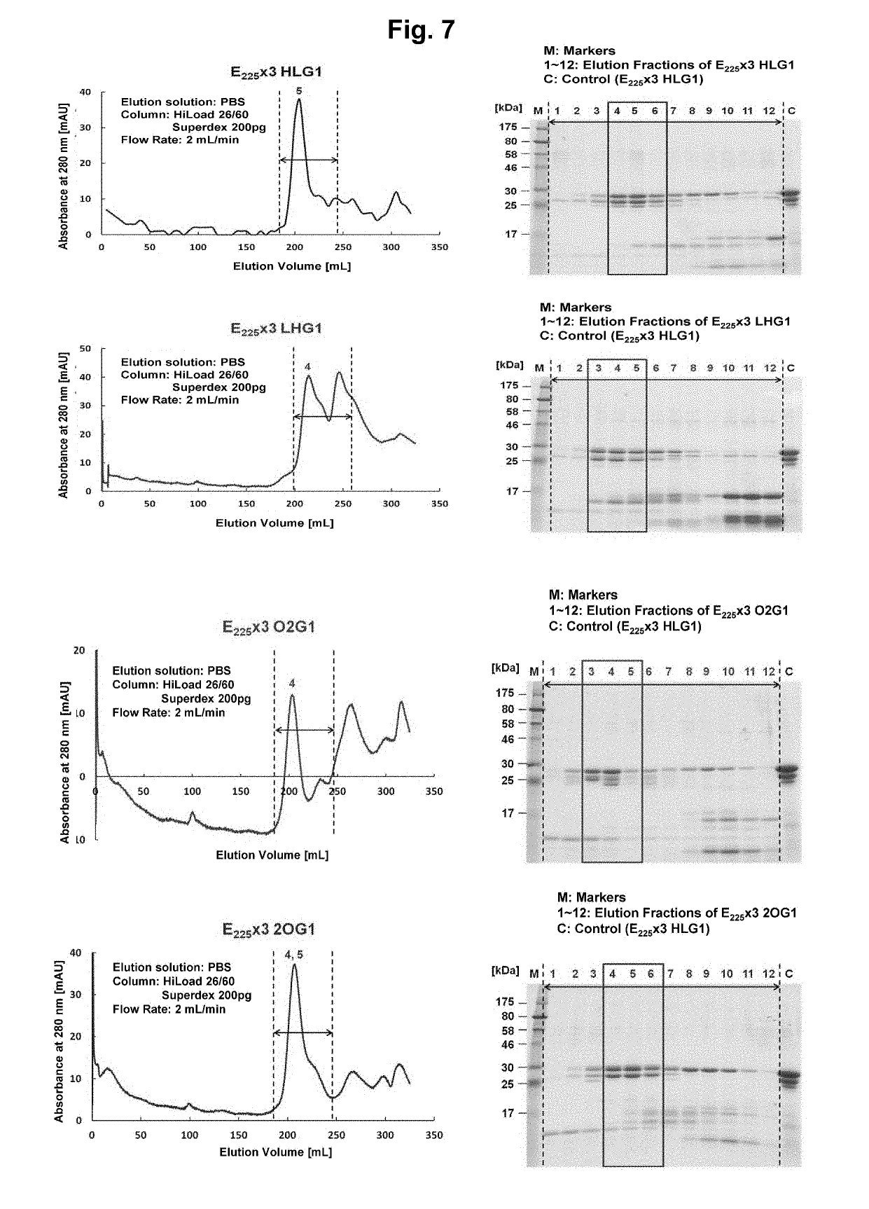

As a result, since a fraction with a relatively high purity could be obtained for E₂₂₅x3 2OG1 and E₂₂₅x3 O₂G1 as well, the 300 mM imidazole elution fraction was further purified by means of gel filtration chromatography. The results of the purification with the gel filtration chromatography of each E₂₂₅x3 are shown in FIG. 7. After the final purification with gel filtration chromatography followed by filter sterilization was done, a yield of each E₂₂₅x3 Db per 1L of the culture medium was calculated, and compared that of Ex3 Db. It is shown that the yields of E₂₂₅x3 Db are much lower than those of Ex3 Db when prepared in the same culture conditions.

TABLE 7
Comparison of yield between various E₂₂₅x3 Db and Ex3 Db

| E₂₂₃x3 Db | Yield [mg/L] | Ex3 Db | Yield [mg/L] |
|---|---|---|---|
| HLG1 | 0.103 | HLG1 | 3.2 |
| LHG1 | 0.046 | LHG1 | 0.7 |
| O2G1 | 0.034 | O5G1 | 2.1 |
| 2OG1 | 0.056 | 5OG1 | 2.2 |

### Comparison of Four Kinds of E₂₂₅x3 Db in Cytotoxicity

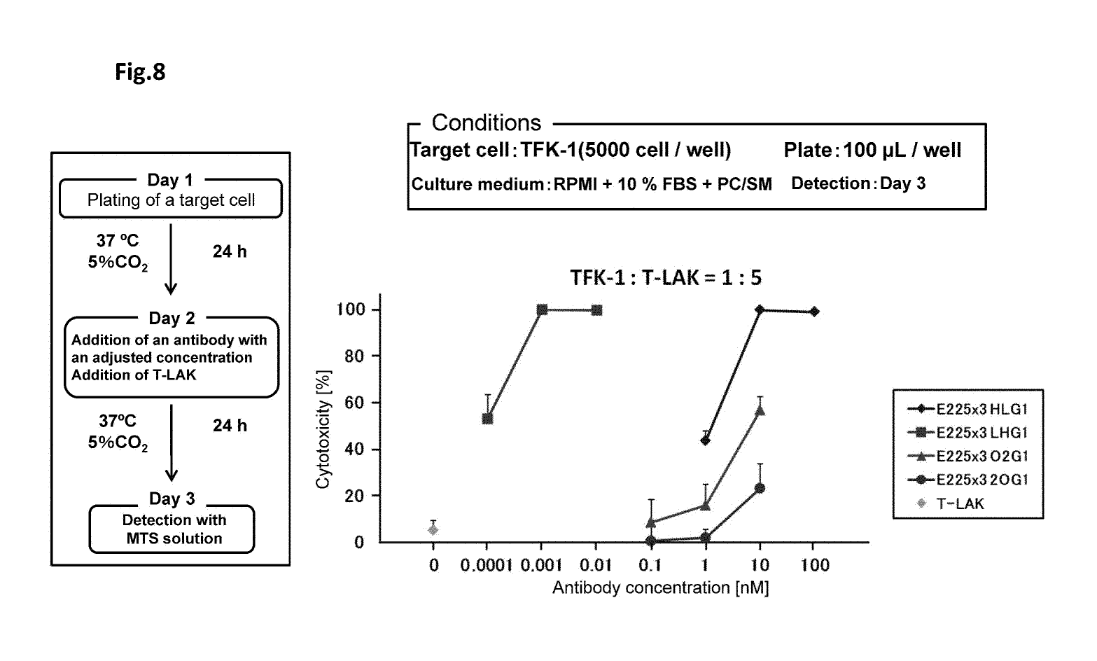

The difference was observed in cytotoxicity among the Ex3 Dbs having difference orientation. Accordingly, MTS assay was carried out with respect to each of the E₂₂₅x3 Dbs using the TFK-1 as a target cell and T-LAK as an effector cell (FIG. 8). As a result, the order in cytotoxicity of the four kinds of the E₂₂₅x3 Dbs having different orientation was LHG1>HLG1.>O2G1>2OG1. It shows that the cytotoxicity of the LHG1 was strongest like in Ex3 Db, so that the functional orientation of LHG1 is common between the anti-EGFR antibodies 528 and 225. On the hand, the E₂₂₅x3 Dbs showed a different tendency from Ex3 Dbs in cytotoxicity among the other orientations. Accordingly, functional analysis was carried out with various methods in order to study the reasons for such difference of E₂₂₅x3 Dbs in cytotoxicity.

### Comparison of Affinity of Four Kinds of E₂₂₅x3 Db for EGFR

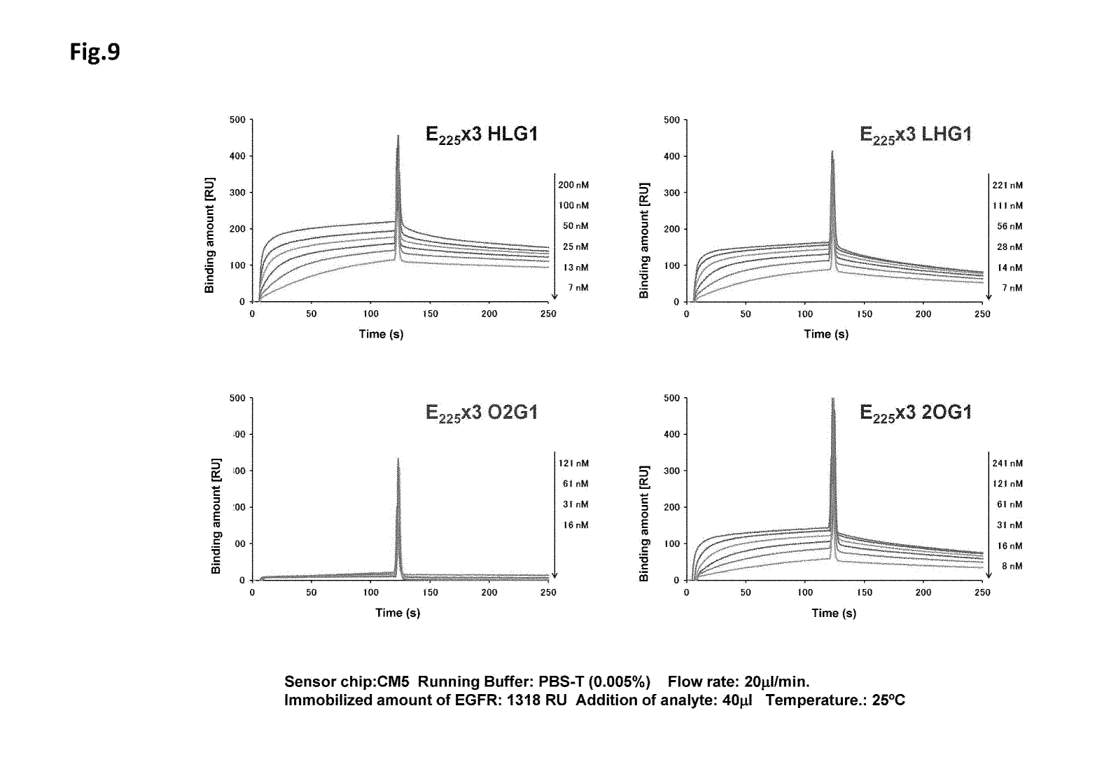

Affinity for EGFR (immobilized amount: 1318 RU) was evaluated by means of SPR (J Biol Chem. 2010 Jul 2:285 (27): 20844-9) in order to study the correlation between the affinity for the antigen and the difference in cytotoxicity of E₂₂₅x3 Dbs. The results of SPR determination are shown in FIG. 9, and binding and dissociation constants calculated based on them are shown in Table 8.

TABLE 8

| | Immobilized | | | | |
|---|---|---|---|---|---|
| | amount of | | | | |
| | EGFR [RU] | Ka [1/(M/s)] | Kd [1/s] | K_{A} [1/M] | K_{D} [M] |
| E₂₂₃x3 HLG1 | 1318 | 8.76 × 10⁵ | 1.66 × 10⁻³ | 5.27 × 10⁸ | 1.90 × 10⁻⁹ |
| E₂₂₃x3 LHG1 | 1318 | 8.16 × 10⁵ | 4.29 × 10⁻³ | 1.90 × 10⁸ | 5.26 × 10⁻⁹ |
| E₂₂₃x3 O2G1 | 1318 | 4.02 × 10² | 1.41 × 10⁻³ | 2.87 × 10⁵ | 3.48 × 10⁻⁸ |
| E₂₂₃x3 2OG1 | 1318 | 3.93 × 10⁵ | 3.96 × 10⁻³ | 9.92 × 10⁷ | 1.01 × 10⁻⁸ |

Ka: Binding rate factor;
Kd: Dissociation rate constant;
K_{A}: Binding equilibrium constant;
K_{D}: Dissociation equilibrium constant

The values of binding equilibrium constants are HLG1>LHG1>2OG1>>O2G1, indicating that there was no correlation between the cytotoxicity and affinity for EGFR. It has therefore been revealed that the difference in the activity of various E₂₂₃x3 Dbs is not attributed to their affinity for EGFR. It was also revealed that the affinity of O2G1 for EGFR was extremely lower than that of the other orientations.

### Comparison of Bridge-Building Capability of Four Kinds of E₂₂₅x3 Db for EGFR

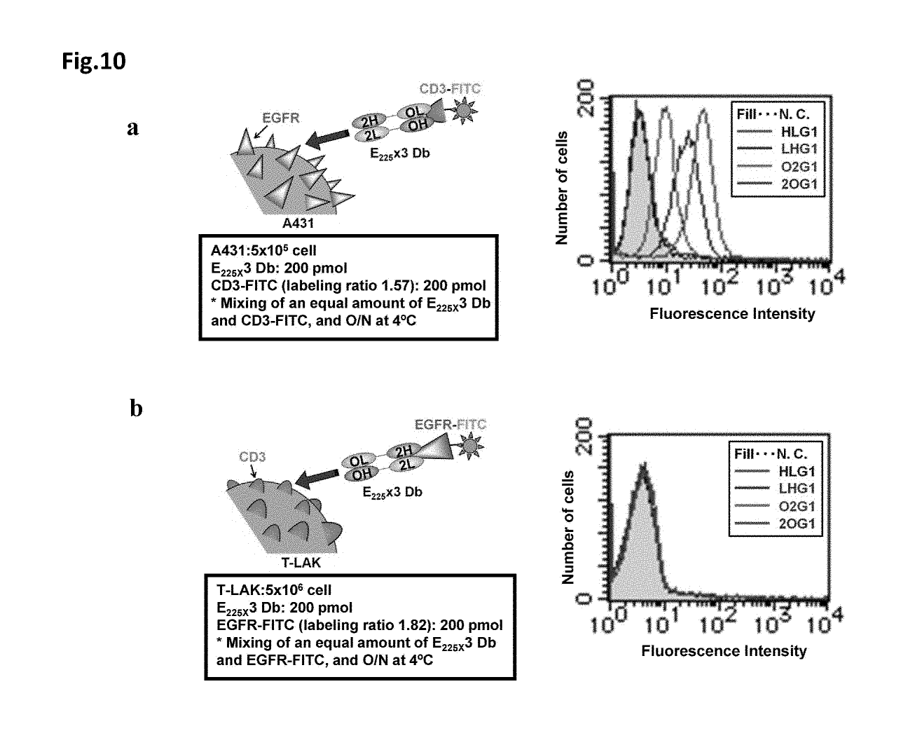

According to the method known for those skilled in the art such as that described in Example 2 of Patent Document 5, bridge-building capability of four kinds of E₂₂₅x3 Db was compared by means of Flow cytometry (FCM). An equal amount (mol) of each E₂₂₅x3 and CD3-FITC were mixed, and binding to EGFR on the surface of a human epidermoid cancer cell A431 (ATCC No. CRL-1555) was detected (FIG. 10a). An equal amount (mol) of each E₂₂₅x3 and EGFR-FITC were mixed, and binding to CD3 on the surface of the cytotoxic T cell, T-LAK (CD3+) was detected (FIG. 10b)

The binding of the three E₂₂₅x3 Db-CD3 complexes with EGFR on the surface of A431 was detected except for 2OG1, the fluoresce intensity being LHG1>HLG1>O2G1. This suggests that the superiority in the bridge-building capability of E₂₂₅x3 LHG1 contributes to its high cytotoxicity. Correlation between the bridge-building capability and cytotoxicity was recognized in E₂₂₅x3 Db. On the other hand, binding of the E₂₂₅x3 Db-EGFR complex with CD3 on the surface of T-LAK was not detected in any orientation. The binding of E₂₂₅x3 Db alone with the CD3 on the surface of T-LAK was observed. It is therefore speculated that a steric hindrance due to the existence of TCR and the like on the cell surface could affect the bridge-building capability of the E₂₂₅x3 Db-EGFR complex to bind with CD3.

### Comparison of the Capability of Inducing Cytokine Secretion of Four Kinds of E₂₂₅x3 Db for EGFR

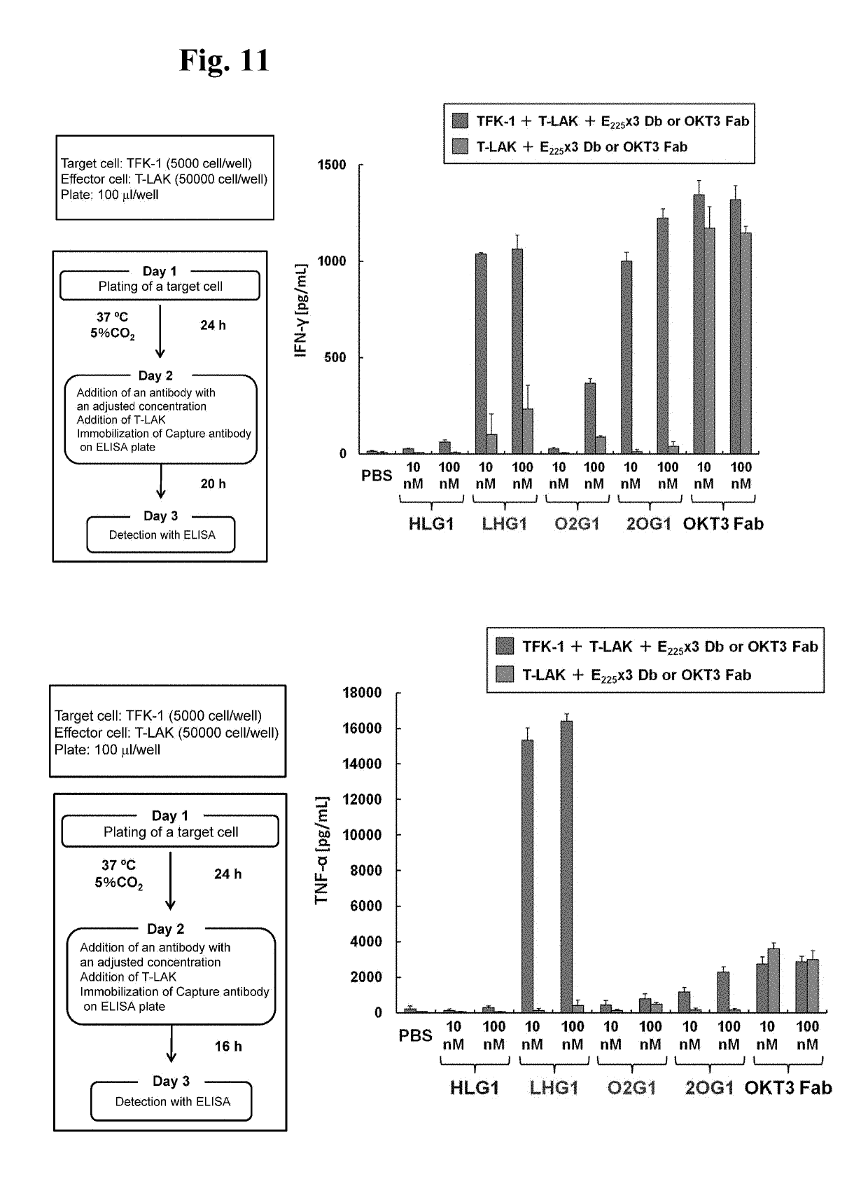

Anti-tumor cytokines were detected with EILSA according to the method known for those skilled in the art (J Biol Chem. 2011 Jan. 21:286 (3): 1812-8) for each E₂₂₅x3 Db in order to study about the correlation with their activities. The results on the detection of IFN-γ and TNF-α in the presence or absence TFK-1, and TKH-1:T-LAK=1:10 are shown in FIG. 11 upper and lower, respectively. A mouse OKT3 Fab was used as a control.

The results in FIG. 11 show that the secretion amount of the cytokines in the co-culture of TFK-1, E₂₂₅x3Db and T-LAK were as follows: IFN-γ: LHG1≈2OG1>O2G1>HLG1, and TNF-α: LHG1>2OG1>O2G1>HLG1. LHG1 having an advantageous orientation for the high activity and bridge-building capability is most superior in inducing secretion of the antitumor cytokines. These results suggested that the induction of cytokine secretion initiated by building a bridge between the target cell and the effector cell, that is to say, the activation of T-LAK is the most important mechanism for the cytotoxicity of E225x3 Db. Since the secretion of the cytokines were very low in the absence of TFK-1, it is also speculated both the target cell and effector cell are involved in the induction of cytokine secretion. Mouse OKT3 Fab promoted the secretion of cytokines irrespective of the presence of TFK-1.

### EXAMPLE 5

### Evaluation of Cytotoxicity of E₂₂₅x3 Db and Ex3 Db

Since both E₂₂₅x3 Db and Ex3 Db target EGFR and CD3, it would be possible and important to compare their activities in the cytotoxicity test using the common target cell and effector cell. Thus, cytotoxicity of E₂₂₅x3 Db and Ex3 Db was compared in vitro and in vivo.

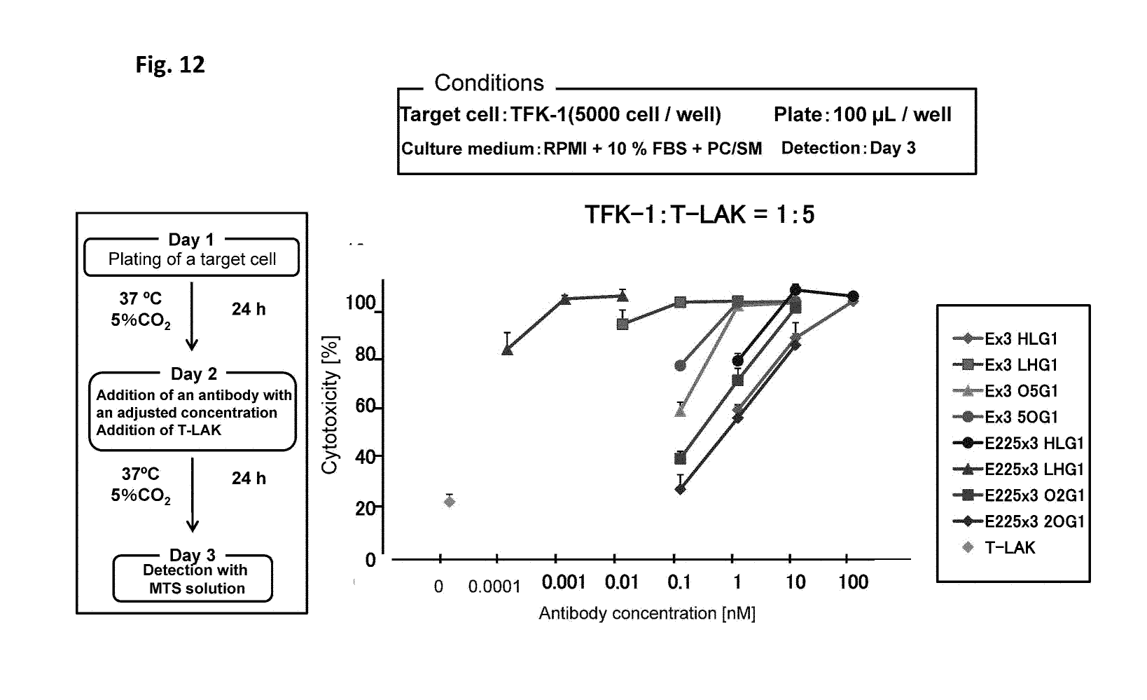

FIG. 12 shows comparison of the cytotoxicity obtained in MTS assay using the four kinds of E₂₂₅x3 Db and Ex3 Db having the different orientation.

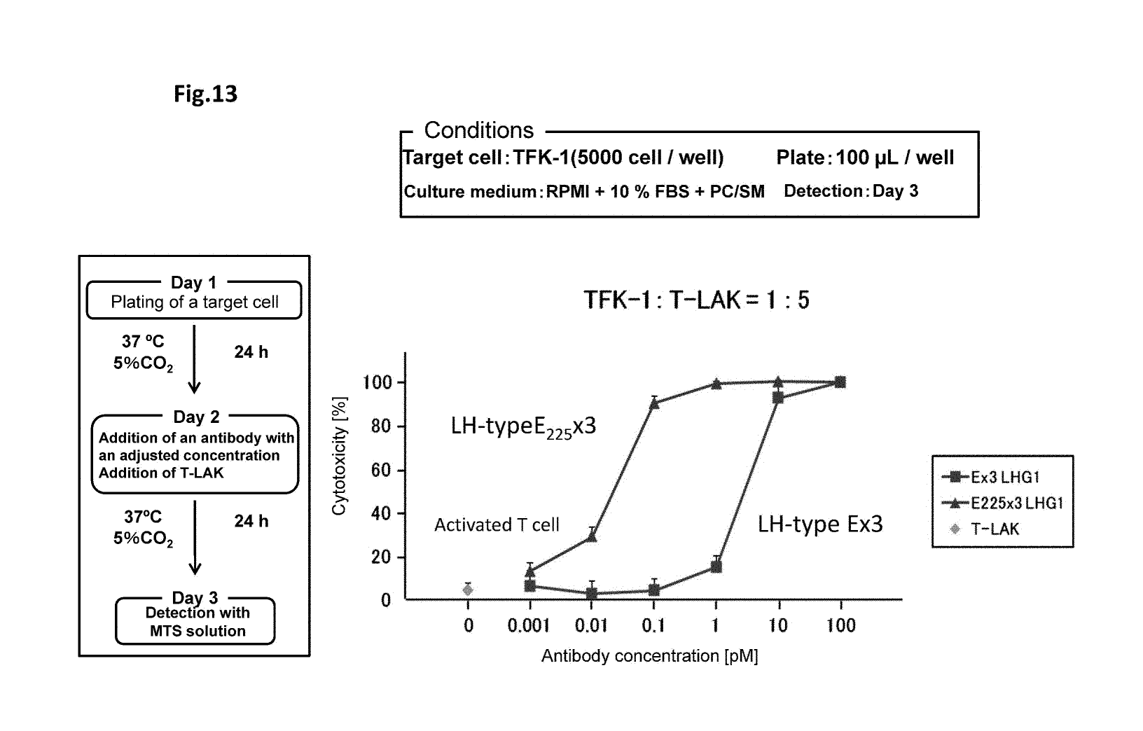

FIG. 13 shows comparison of the cytotoxicity of E₂₂₅x3 LHG1 and Ex3 LHG1 in MTS assay, indicating that the cytotoxicity of E₂₂₅x3 LHG1 is about 100 times higher than that of Ex3 LHG1.

### EXAMPLE 6

### Comparison of the Cytotoxicity In Vivo of E₂₂₅x3 LHG1 and Ex3 LHG1

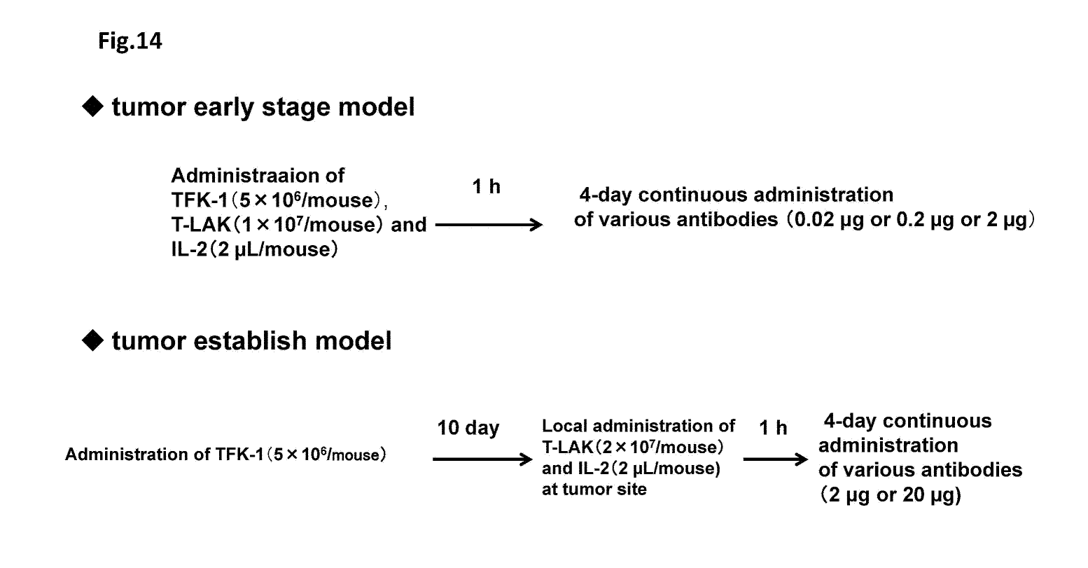

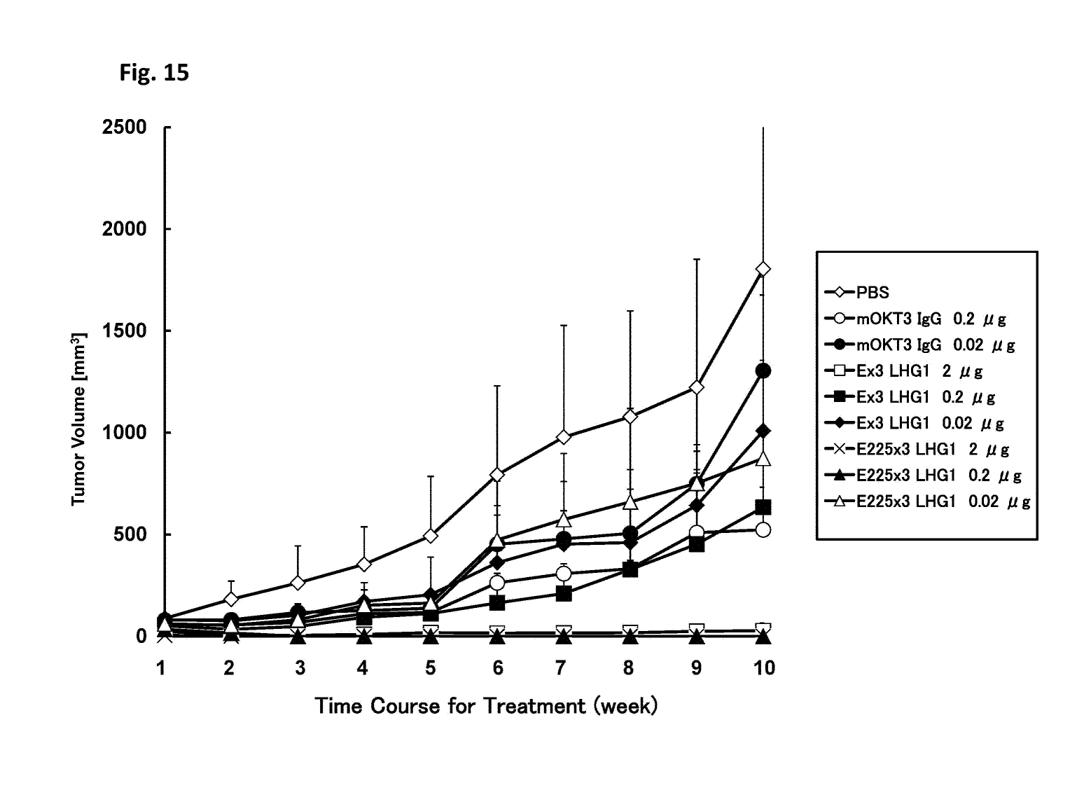

As shown in Example 5, the four kinds of E₂₂₅x3 Db and Ex3 Db had the cytotoxicity in vitro. Thus, evaluation of in vivo activity was carried out in a tumor early stage model using a SCID mouse (FIG. 14). In addition to a negative control (administration of PBS alone), mOKT3 IgG was used as a control. As shown in FIG. 15, while an effective growth-inhibition of tumor could be recognized only when an administration of Ex3 Db has been increased up to 2 μg, a perfect growth-inhibition of tumor was recognized even at the administration of 0.2 μg, indicating the correlation of the superiority of the anti-tumor activities between in vitro and in vivo.

### INDUSTRIAL APPLICABILITY

The present invention has revealed that even if the target antigen is the same and the binding properties are similar with each other such as between the 528 and 225 antibodies, their functions could greatly change in the form of the bispecific antibodies. It accordingly suggests that there is a possibility that a more functional bispecific antibody can be developed though studying a kind of an antibody against a target antigen.

1. A bispecific antibody, comprising a variable region of the light chain (2L: SEQ ID NO:2) and a variable region of the heavy chain (2H: SEQ ID NO:4) of an anti-human EGF receptor 1 antibody 225, and a humanized variable region of the light chain (OL: SEQ ID NO:6) and a humanized variable region of the heavy chain (OH: SEQ ID NO:8) of an anti-CD3 antibody OKT.

2. The antibody according to claim 1, which is a diabody-type bispecific antibody.

3. The antibody according to claim 1, wherein the variable region of the light chain is located at an N-end side of the variable region of the heavy chain (LH-type) in each polypeptide.

4. The antibody according to claim 1, which is a tandem-type single chain antibody (scFv) having the structure represented by (2L2H)-(peptide linker)-(OHOL)

5. The antibody according to claim 1, which further comprises a hinge region and Fc region.

6. The antibody according to claim 1, which is a bispecific antibody for human EGF receptor 1 and CD3.

7. A single-chain polypeptide constituting the antibody of claim 1.

8. A nucleic acid molecule encoding the polypeptide of claim 7.

9. A replicable cloning vector or an expression vector containing the nucleic acid molecule of claim 8.

10. The vector of claim 9, which is a plasmid vector.

11. A host cell transformed with the vector of claim 9.

12. The hose cell of claim 11, which is a mammalian cell.

13. A method for the production of the antibody of claim 1, comprising culturing a host cell transformed with a replicable cloning vector or an expression vector containing a nucleic acid molecule encoding a single-chain polypeptide constituting said antibody, to express the nucleic acid molecule, and collecting and purifying the single-chain polypeptides constituting said antibody, assembling the resulting single-chain polypeptides, and separating and collecting said antibody of thus formed.

14. A pharmaceutical composition comprising the antibody of claim 1 as an active ingredient.

15. The pharmaceutical composition of claim 14 for use in eliminating, hurting, damaging and/or reducing tumor cells.
